(19) United States
(12) Patent Application Publication
(10) Pub. No.: US 2014/0276212 A1
(43) Pub. Date: Sep. 18, 2014

(54) SYSTEMS, DEVICES, AND METHODS INCLUDING INTESTINAL MICROBIAL FLORA MAPPING

(71) Applicant: ELWHA LLC, Bellevue, WA (US)

(72) Inventors: Roderick A. Hyde, Redmond, WA (US); Lowell L. Wood, JR., Bellevue, WA (US)

(21) Appl. No.: 13/831,000

(22) Filed: Mar. 14, 2013

(51) Int. Cl. A61B 10/00; A61N 7/00; A61M 31/00

(52) U.S. Cl. CPC A61B 10/00; A61M 31/00; A61N 7/00
USPC 600/570; 607/133; 604/285; 600/562; 601/2

(57) ABSTRACT

Systems, devices, and methods are described for providing, among other things, devices operable to acquire intestinal microbial flora samples, to map intestinal microbial flora, to identify microbes present in an individual's digestive tract, to delivery intestinal microbial flora compositions, to register a microbial flora collection events, to register microbial flora-seeding events, or the like.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None

### RELATED APPLICATIONS

None

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

In an aspect, the present disclosure is directed to, among other things, an electronic suppository system including an electronic suppository egress module. In an embodiment, the electronic suppository egress module includes circuitry configured to control an egress process associated with egress of an electronic suppository from an intestinal tract. In an embodiment, the electronic suppository egress module includes circuitry configured to actuate an egress process associated with egress of an electronic suppository responsive to at least one input indicative of an onset of a defecation event. In an embodiment, the electronic suppository egress module includes circuitry configured to actuate an egress process associated with egress of an electronic suppository responsive to at least one input indicative of a peristaltic contraction in an intestinal tract. In an embodiment, the electronic suppository egress module includes circuitry configured to actuate an egress process associated with egress of an electronic suppository responsive to at least one input indicative of a peristaltic reflex process. In an embodiment, the electronic suppository egress module includes circuitry configured to actuate an egress process associated with egress of an electronic suppository responsive to a target schedule.

In an aspect, the present disclosure is directed to, among other things, an article of manufacture including a non-transitory signal-bearing medium bearing one or more instruction for causing a computing device to control an egress process associated with egress of an electronic suppository from an intestinal tract. In an embodiment, the article of manufacture includes a non-transitory signal-bearing medium bearing one or more instruction for causing a computing device to actuate electrical stimulation of one or more regions of intestinal mucosal surface proximate the electronic suppository. In an embodiment, the article of manufacture includes a non-transitory signal-bearing medium bearing one or more instruction for causing a computing device to actuate one or more transducers to acoustically stimulate a region of intestinal mucosal surface proximate the electronic suppository. In an embodiment, the article of manufacture includes a non-transitory signal-bearing medium bearing one or more instruction for causing a computing device to actuate one or more surface contact elements associated with an electronic suppository to physically contact one or more regions of intestinal mucosal surface proximate the electronic suppository and to mechanically stimulate one or more regions of an intestinal mucosal surface.

In an aspect, the present disclosure is directed to, among other things, an electronic suppository system including a flora-sampling module. In an embodiment, the flora-sampling module includes circuitry configured to actuate the collection of at least one intestinal microbial flora sample via a plurality of collection ports. In an embodiment, the electronic suppository system includes a flora-mapping module. In an embodiment, the flora-mapping module includes circuitry configured to generate flora collection information associated with at least one intestinal microbial flora sample.

In an aspect, the present disclosure is directed to, among other things, an electronic suppository including a housing structure and a flora-sampling module having a plurality of collection ports. In an embodiment, the plurality of collection ports is arranged and configured to provide fluidic communication between an interior environment of the housing structure and an exterior environment. In an embodiment, the flora-sampling module is configured to acquire an intestinal microbial flora sample. In an embodiment, the electronic suppository includes at least one reservoir operably coupled via at least one flow path to one or more of the plurality of collection ports and configured to receive and store an intestinal microbial flora sample. In an embodiment, the flora-sampling module includes circuitry configured to actuate the collection of at least one intestinal microbial flora sample via at least one of the plurality of collection ports. In an embodiment, the flora-sampling module includes circuitry configured to actuate the storing of at least one intestinal microbial flora sample within one or more reservoirs.

In an embodiment, the electronic suppository includes a flora-mapping module including circuitry configured to generate flora collection information associated with at least one intestinal microbial flora sample collected by an electronic suppository. In an embodiment, the electronic suppository includes a suppository navigation module including circuitry configured to actuate advancement of the electronic suppository along an interior of an intestinal tract responsive to one or more navigation inputs.

In an aspect, the present disclosure is directed to, among other things, an electronic suppository including a housing structure having at least one payload module and at least one power module. In an embodiment, the payload module includes a plurality of collection ports arranged to provide fluidic communication between an interior environment of the housing structure and an exterior environment, and configured to acquire an intestinal microbial flora sample. In an embodiment, the payload module includes at least one reservoir operably coupled via at least one flow path to one or more of the plurality of collection ports and configured to receive and store an intestinal microbial flora sample. In an embodiment, a power module includes a power source and a communication module including circuitry configured to communicate with a remote enterprise and to receive control command information from the remote enterprise. In an embodiment, a power module includes a power source and at least one of a receiver, a transmitter, and a transceiver operable to communicate with a remote enterprise and to receive control command information from the remote enterprise.

In an aspect, the present disclosure is directed to, among other things, an intestinal-microbial-flora-seeding suppository including a housing structure and a plurality of microbial flora reservoirs received within the housing structure. In an embodiment, the intestinal-microbial-flora-seeding suppository includes a plurality of selectively actuatable delivery ports operably coupled to one or more of the plurality of microbial flora reservoirs. In an embodiment, the plurality of selectively actuatable delivery ports is operable to deliver an intestinal microbial flora composition received in one more of the plurality of microbial flora reservoirs to an exterior environment. In an embodiment, the intestinal-microbial-flora-seeding suppository includes a microbial flora delivery module including circuitry configured to actuate the delivery of the intestinal microbial flora composition from one or more the plurality of microbial flora reservoirs to an exterior environment of the housing structure responsive to user-specific microbial flora seeding information.

In an aspect, the present disclosure is directed to, among other things, a method of delivering microbial flora within an intestinal tract. In an embodiment, the method includes registering at least a first intestinal anatomical target along an intestinal tract to user-specific intestinal registration information. In an embodiment, registering the first intestinal anatomical target includes determining a plurality of references points along the intestinal tract and registering the first intestinal anatomical target to the plurality of references points. In an embodiment, the method includes delivering, via an electronic suppository, a microbial flora composition to the first intestinal anatomical target. In an embodiment, the method includes registering at least a second intestinal anatomical target along an intestinal tract to user-specific intestinal registration information. In an embodiment, the method includes delivering, via an electronic suppository, a microbial flora composition to the second intestinal anatomical target.

In an aspect, the present disclosure is directed to, among other things, an article of manufacture including a non-transitory signal-bearing medium bearing one or more instruction for causing a computing device to registering at least a first intestinal anatomical target along an intestinal tract to user-specific intestinal registration information. In an embodiment, the article of manufacture includes a non-transitory signal-bearing medium bearing one or more instruction for causing a computing device to deliver, via an electronic suppository, a microbial flora composition to the first intestinal anatomical target. In an embodiment, the article of manufacture includes a non-transitory signal-bearing medium bearing one or more instruction for causing a computing device to deliver, via an electronic suppository, a microbial flora composition to the first intestinal anatomical target responsive to a comparison to a comparison between the first intestinal anatomical target along an intestinal tract and the user-specific intestinal registration information. In an embodiment, the article of manufacture includes a non-transitory signal-bearing medium bearing one or more instruction for causing a computing device to deliver, via an electronic suppository, a microbial flora composition to the first intestinal anatomical target responsive to registration information indicative of a target location.

In an embodiment, the article of manufacture includes a non-transitory signal-bearing medium bearing one or more instruction for causing a computing device to register at least a second intestinal anatomical target. In an embodiment, the article of manufacture includes a non-transitory signal-bearing medium bearing one or more instruction for causing a computing device to deliver, via the electronic suppository, a microbial flora composition to the second anatomical target. In an embodiment, the article of manufacture includes a non-transitory signal-bearing medium bearing one or more instruction for causing a computing device to deliver, via an electronic suppository, a microbial flora composition to the second intestinal anatomical target responsive to a comparison to a comparison between the first intestinal anatomical target along an intestinal tract and the user-specific intestinal registration information. In an embodiment, the article of manufacture includes a non-transitory signal-bearing medium bearing one or more instruction for causing a computing device to deliver, via an electronic suppository, a microbial flora composition to the second intestinal anatomical target responsive to registration information indicative of a target location.

In an aspect, the present disclosure is directed to, among other things, an ingestible flora-sampling device including a flora-sampling module and a flora-mapping module. In an embodiment, the flora-sampling module includes circuitry configured to actuate collection of at least one intestinal microbial flora sample via a plurality of collection ports. In an embodiment, the flora-sampling module includes circuitry configured to actuate the collection of a plurality of intestinal microbial flora samples, a plurality of fluid samples from intestinal mucosa, responsive to a patient-specific collection protocol. In an embodiment, the flora-mapping module includes circuitry configured to generate flora collection information associated with at least one intestinal microbial flora sample.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a perspective view of electronic suppository system according to one embodiment.

FIG. 2 is a perspective view of electronic suppository system according to one embodiment.

FIG. 3 is a perspective view of an ingestible device according to one embodiment.

FIGS. 4A and 4B show a flow diagram of an article of manufacture according to one embodiment.

FIG. 5 shows a flow diagram of an article of manufacture according to one embodiment.

FIG. 6 shows a flow diagram of a method according to one embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Microbial communities including bacteria, microbes, single-cell eukaryotes viruses, etc., can be found at various different environments and locations on the human body, including, for example, nasal passages, oral cavities, skin, gastrointestinal tract, urogenital tract, etc. These microbial communities may play a significant role in mammalian disease, health, immunity, nutrition, etc.

In an embodiment, technologies and methodologies are provided for mapping intestinal microbial flora of a biological subject, seeding intestinal microbial flora of a biological subject, quantifying intestinal microbial flora of a biological subject, qualifying intestinal microbial flora of a biological subject, or the like. For example, FIG. 1 shows a system 100 (e.g., electronic suppository system, an intestinal-microbial-flora-seeding system, an intestinal-microbial-flora-mapping system, an intestinal microbial flora sampling system, etc.), in which one or more methodologies or technologies can be implemented such as, for example, delivering microbial flora within an intestinal tract to one or more target locations, registering one or more intestinal anatomical targets, generating microbial flora delivery registration information, generating flora collection information associate, mapping microbes present in an individual's digestive tract, identifying microbes present in an individual's digestive tract, evaluating intestinal microflora (e.g., qualifying intestinal microflora, quantifying intestinal microflora, detecting intestinal microflora, etc.) or the like.

In an embodiment, the system 100 includes an electronic suppository 101 having an electronic suppository egress module 102 configured to control an egress process associated with egress of an electronic suppository 101 from an intestinal tract. For example, in an embodiment, the electronic suppository egress module 102 includes circuitry configured to generate one or more egress commands (e.g., electronic inputs, outputs, instructions, or the like) to one or more module to initiate, actuate, activate, etc., at least one egress protocol.

In an embodiment, a module includes, among other things, one or more computing devices such as a processor (e.g., a microprocessor, a quantum processor, qubit processor, etc.), a central processing unit (CPU), a digital signal processor (DSP), an application-specific integrated circuit (ASIC), a field programmable gate array (FPGA), or the like, or any combinations thereof, and can include discrete digital or analog circuit elements or electronics, or combinations thereof. In an embodiment, a module includes one or more ASICs having a plurality of predefined logic components. In an embodiment, a module includes one or more FPGAs, each having a plurality of programmable logic components.

In an embodiment, the electronic suppository egress module 102 includes a module having one or more components operably coupled (e.g., communicatively, electromagnetically, magnetically, ultrasonically, optically, inductively, electrically, capacitively coupled, wirelessly coupled, or the like) to each other. In an embodiment, a module includes one or more remotely located components. In an embodiment, remotely located components are operably coupled, for example, via wireless communication. In an embodiment, remotely located components are operably coupled, for example, via one or more communication modules, receivers 104, transmitters 106, transceivers 108, or the like. In an embodiment, the electronic suppository egress module 102 includes a module having one or more routines, components, data structures, interfaces, and the like.

In an embodiment, a module includes memory 110 that, for example, stores instructions or information. For example, in an embodiment, at least one control module includes memory 110 that stores control command information, electronic suppository status information, flora collection information, intestinal travel path markings information, intestinal travel-route status information, navigation plan information, patient identification information, registration information, suppository travel route-status information, user-specific intestinal registration information, user-specific microbial flora seeding information, etc.

Non-limiting examples of memory 110 include volatile memory (e.g., Random Access Memory (RAM), Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like), persistent memory, or the like. Further non-limiting examples of memory include Erasable Programmable Read-Only Memory (EPROM), flash memory, or the like. In an embodiment, memory is coupled to, for example, one or more computing devices by one or more instructions, information, or power buses. In an embodiment, the electronic suppository egress module 102 includes memory that stores, for example, user identification information, suppository travel-route status information, suppository route registration information, or the like. In an embodiment, electronic suppository egress module 102 includes memory 110 that stores, for example, suppository tracking information, intestinal tract registration information, suppository control command information, user-specific microbial flora information, or the like.

In an embodiment, a module includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, a module includes one or more user input/output components that are operably coupled to at least one computing device configured to control (electrical, electromechanical, software-implemented, firmware-implemented, or other control, or combinations thereof) at least one parameter associated with, for example, controlling egress of an electronic suppository 101.

In an embodiment, a module includes a computer-readable media drive or memory slot that is configured to accept signal-bearing medium (e.g., computer-readable memory media, computer-readable recording media, or the like). In an embodiment, a program for causing a system to execute any of the disclosed methods can be stored on, for example, a computer-readable recording medium, a signal-bearing medium, or the like. Non-limiting examples of signal-bearing media include a recordable type medium such as a magnetic tape, floppy disk, a hard disk drive, a Compact Disc (CD), a Digital Video Disk (DVD), Blu-Ray Disc, a digital tape, a computer memory, or the like, as well as transmission type medium such as a digital or an analog communication medium (e.g., a fiber optic cable, a waveguide, a wired communications link, a wireless communication link (e.g., receiver 104, transmitter 106, transceiver 108, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In an embodiment, the system 100 includes an electronic suppository egress module 102 having circuitry configured to control an egress process associated with egress of an electronic suppository 101 from an intestinal tract. In an embodiment, the electronic suppository egress module 102 includes circuitry configured to actuate an egress process associated with egress of an electronic suppository 101 responsive to at least one input indicative of an onset of a defecation event. For example, during operation, upon sensing a muscular contraction associated with a defecation reflex, the electronic suppository egress module 102 actuates an egress process associated with egress of an electronic suppository 101. In an embodiment, this can include delivering at least one lubricating material from or to an outer surface on an electronic suppository 101. In an embodiment, the electronic suppository egress module 102 includes circuitry configured to actuate an egress process associated with egress of an electronic suppository 101 responsive to at least one measurand from one or more sensors 154 indicative of a contraction of a pelvic diaphragm.

In an embodiment, the electronic suppository egress module 102 includes circuitry configured to actuate an egress process associated with egress of an electronic suppository 101 responsive to at least one measurand from one or more sensors 154 indicative of a contraction of one or more expiratory chest muscles. In an embodiment, the electronic suppository egress module 102 includes circuitry configured to actuate an egress process associated with egress of an electronic suppository 101 responsive to at least one measurand from one or more sensors 154 indicative of a diaphragm contraction. In an embodiment, the electronic suppository egress module 102 includes circuitry configured to actuate an egress process associated with egress of an electronic suppository 101 responsive to at least one measurand from one or more sensors 154 indicative of a contraction of one or more abdominal wall muscles. In an embodiment, the electronic suppository egress module 102 includes circuitry configured to actuate an egress process associated with egress of an electronic suppository 101 responsive to at least one measurand from one or more sensors 154 indicative of a pressure on the digestive tract.

In an embodiment, the electronic suppository egress module 102 includes circuitry configured to activate egress of an electronic suppository responsive to at least one datum indicative of a completion of a task by the suppository 101.

In an embodiment, the electronic suppository egress module 102 includes circuitry configured to activate egress of an electronic suppository responsive to at least on datum indicative of a compliance with an electronic suppository capture protocol. In an embodiment, the electronic suppository egress module 102 includes circuitry configured to request authorization to egress an electronic suppository 101. For example, in an embodiment, the electronic suppository egress module 102 is operable to activate egress of an electronic suppository 101 based on a response from a request to egress indicative of an authorization to egress. In an embodiment, the electronic suppository egress module 102 is operable to deactivate an egress process of the electronic suppository 101 based on a response from a request to egress indicative of no authorization to egress.

In an embodiment, the electronic suppository egress module 102 includes circuitry configured to actuate an egress process associated with egress of an electronic suppository 101 responsive to at least one measurand from one or more sensors 154 indicative of a change in pressure within a portion of an intestinal tract. In an embodiment, the electronic suppository egress module 102 includes circuitry configured to actuate an egress process associated with egress of an electronic suppository 101 responsive to at least one measurand from one or more sensors 154 indicative of one or more peristaltic contractions. In an embodiment, the electronic suppository egress module 102 includes circuitry configured to actuate an egress process associated with egress of an electronic suppository 101 responsive to at least one measurand from one or more sensors 154 indicative of peristaltic contractions in an intestinal tract.

In an embodiment, the electronic suppository egress module 102 includes circuitry configured to actuate an egress process associated with egress of an electronic suppository 101 responsive to at least one input indicative of a peristaltic reflex process. For example, in an embodiment, the electronic suppository egress module 102 includes circuitry configured to actuate an egress process associated with egress of an electronic suppository 101 responsive to at least one input indicative of a peristaltic contraction in an intestinal tract. In an embodiment, the electronic suppository egress module 102 includes circuitry configured to actuate an egress process associated with egress of an electronic suppository 101 responsive to a time-based protocol.

In an embodiment, the system 100 includes a lubricant supply module 118 operably coupled to the electronic suppository egress module 102.

In an embodiment, the lubricant supply module 118 includes circuitry configured to activate delivery of at least one lubricating material from the one or more reservoirs 114, to an outer surface on an electronic suppository 101. Non-limiting examples of lubricating materials include paraffin, glycerin, mineral oil, petroleum jelly, and the like. In an embodiment, an electronic suppository 101 includes one or more the lubricant supply modules 118 adapted to control delivery of at least one lubricating material from at least one reservoir 114, in a spatially patterned distribution, by actuating one or more ports 116, valves (e.g., adjustable pressure valves, mono-pressure valves, mechanical valves, electro-mechanical values, programmable valves, needle valves, valve mechanisms (e.g., ball-in-cone mechanism), or the like), flow paths (e.g., fluid-flow passageways, conduits, channels, lumens, or the like), etc. For example, in an embodiment, the reservoir 114 has release ports 116 and the lubricant supply module 118 controls the opening and closing of the release ports 116. In an embodiment, during operation, the lubricant supply module 118 controls the delivery of at least one lubricating material from reservoir 114 to an exterior of an electronic suppository 101 by controlling the actuation one or more ports 116, valves, flow path inlets, flow path outlets, or the like.

In an embodiment, the lubricant supply module 118 includes circuitry operable to electronically controls the operation of one or more ports 116, valves, flow path inlets, flow path outlets, or the like. In an embodiment, the lubricant supply module 118 includes circuitry having one or more components operably coupled (e.g., communicatively, electromagnetically, magnetically, ultrasonically, optically, inductively, electrically, capacitively coupled, or the like) to each other. In an embodiment, circuitry includes one or more remotely located components. In an embodiment, remotely located components are operably coupled via wireless communication. In an embodiment, remotely located components are operably coupled via one or more one or more receivers 104, transmitters 106, transceivers 108, or the like.

In an embodiment, circuitry includes, among other things, one or more computing devices such as a processor (e.g., a microprocessor), a central processing unit (CPU), a digital signal processor (DSP), an application-specific integrated circuit (ASIC), a field programmable gate array (FPGA), or the like, or any combinations thereof, and can include discrete digital or analog circuit elements or electronics, or combinations thereof. In an embodiment, circuitry includes one or more ASICs having a plurality of predefined logic components. In an embodiment, circuitry includes one or more FPGAs having a plurality of programmable logic components.

In an embodiment, circuitry includes one or more memories 110 that, for example, store instructions or data. Non-limiting examples of examples of one or more memories 110 include volatile memory (e.g., Random Access Memory (RAM) 110, Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like), persistent memory, or the like. Further non-limiting examples of one or more memories 110 include Erasable Programmable Read-Only Memory (EPROM), flash memory, or the like. The one or more memories 110 can be coupled to, for example, one or more computing devices by one or more instruction, data, or power buses.

In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, circuitry includes one or more user input/output components that are operably coupled to at least one computing device to control (electrical, electromechanical, software-implemented, firmware-implemented, or other control, or combinations thereof) at least one parameter associated with actuating delivery of at least one lubricating material from at least one reservoir through one or more ports 116, valves, flow paths, etc., to an exterior environment of an electronic suppository 101.

In an embodiment, during operation, reservoirs 114, ports 116, valves, flow paths (e.g., fluid-flow passageways, conduits, channels, lumens, or the like), etc., under the control of the lubricant supply module 118, work in concert to delivery of at least one lubricating material from at least one reservoir 114 through one or more ports 116, valves, flow paths, etc., to an exterior environment of an electronic suppository 101.

In an embodiment, an electronic suppository 101 includes one or more lubricant supply modules 118 including a plurality of spaced-apart release ports 116 adapted to deliver at least one lubricating material in a spatially patterned distribution. In an embodiment, a lubricant supply module 118 is operably coupled to one or more lubricant reservoirs 114 and configured to control at least one of a delivery rate, a delivery amount, a material delivery composition, a port release rate, a port release amount, and a port release pattern, a port release spatially distribution. In an embodiment, the lubricant supply module 118 is operably coupled to one or more lubricant reservoirs 114 and configured to actively control one or more of the plurality of spaced-apart release ports. In an embodiment, at least one lubricant supply module 118 is operably coupled to one or more of the spaced-apart controllable-release ports 116 and configured to control at least one of a port release rate, a port release amount, and a port release pattern associated with a delivery of a lubricating material composition. In an embodiment, at least one processor is operably coupled to the lubricant supply module 118 and configured to control at least one of a port release rate, a port release amount, and a port release pattern associated with the delivery of the lubricating material from the at least one reservoir to an exterior of electronic suppository 101.

In an embodiment, the system 100 includes an intestinal peristalsis induction module 120 operably stimulate, induce, initiate, etc., peristalsis. For example, in an embodiment, the system 100 includes an intestinal peristalsis induction module 120 having circuitry configured to active electrical stimulation of a region of intestinal mucosal surface and induce peristalsis. In an embodiment, the system 100 includes an intestinal peristalsis induction module 120 operably coupled to the electronic suppository egress module 102.

In an embodiment, the system 100 includes an intestinal peristalsis induction module 120 having one or more transducers 122 configured to acoustically stimulate a region of intestinal mucosal surface. In an embodiment, the system 100 includes an intestinal peristalsis induction module 120 operably coupled to the electronic suppository egress module 102, the intestinal peristalsis induction module 120 having one or more surface contact elements 124 configured to physically contact one or more regions of intestinal mucosal surface and to mechanically stimulate a region of intestinal mucosal surface. In an embodiment, the system 100 includes an intestinal peristalsis induction module 120 having a plurality of surface contact elements 124 configured to physically distend one or more regions of intestinal mucosal surface to induce peristalsis. For example, during operation, in an embodiment, two or more adjacent surface contact elements 124 engage a portion of an intestinal mucosal surface as determined by one or more proximity sensors on board the electronic suppository 101. Once adjacent contact elements 124 engage a portion of the intestinal mucosal surface, the peristalsis induction module 120 sends a control signal to an electromechanical component operable to increase a distance 125 between the adjacent contact elements 124 in contact with a portion of intestinal mucosal surface resulting in a distention of a region of intestinal mucosal surface. Stimulate one or more regions of intestinal mucosal surface in this way may induce peristalsis.

In an embodiment, contact elements 124 can be mechanical elements that can be extended or retracted by a mechanical or electrical motor upon activation of the motor by the peristalsis induction module 120. For example, during operation, in an embodiment, the peristalsis induction module 120 includes circuitry configured to activate by a mechanical or electrical motor that causes a plurality of surface contact elements 124 to a mechanical extend and engage portion of intestinal mucosal surface and to mechanically distend portion of intestinal mucosal surface responsive to initiation of an egress protocol.

In an embodiment, the system 100 includes a laxative supply module 126 coupled to the electronic suppository egress module 102, the laxative supply module 126 configured to deliver at least one laxative composition to an outer surface on an electronic suppository 101. For example, in an embodiment, during operation, the laxative supply module 126 controls the delivery of at least one laxative composition from reservoir 114 housed within an electronic suppository 101 by controlling the actuation of one or more ports 116, valves, flow path inlets, flow path outlets, or the like that provide selective fluidic communication between a reservoir 114 within an electronic suppository 101 and an outer surface on the electronic suppository 101. Non-limiting examples of laxatives include carbon dioxide-releasing laxatives, emollient laxatives, hyperosmotic laxatives, lubricant laxatives, saline laxatives, stimulant laxatives, stool softeners, and the like. Further Non-limiting examples of laxatives include bisacodyl, danthron, docusate, glycerin, magnesium hydroxide mineral oil, poloxamer 188, psyllium, saline compositions, senna, sennosides, and the like.

In an embodiment, the electronic suppository egress module 102 includes circuitry configured to actuate delivery of at least one laxative composition responsive to activation of an egress protocol associated with egress of an electronic suppository 101 from an intestinal tract. In an embodiment, the laxative supply module 126 includes circuitry configured to control at least one parameter of, for example, electro-mechanical system including electrical circuitry operably coupled with a transducer (e.g., an actuator, a motor, a piezoelectric crystal, a Micro Electro Mechanical System (MEMS), etc.), that actuates one or more flow control valves between ON and OFF states. In an embodiment, the laxative supply module 126 includes circuitry having at least one of a discrete electrical circuit, an electrical circuitry having at least one integrated circuit, an electrical circuitry having at least one application specific integrated circuit, an electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs.

In an embodiment, the system 100 includes an electronic suppository egress module 102 including circuitry configured to actuate a change in a physical dimension of an electronic suppository 101 to facilitate egress of the electronic suppository from an intestinal tract. In an embodiment, the system 100 includes a defecation detection module 128 operably coupled to the electronic suppository egress module 102. In an embodiment, the defecation detection module 128 includes circuitry configured to generate real-time defecation event information responsive to at least one measurand from one or more sensors 154 associated with a defecation event.

In an embodiment, the defecation detection module 128 is operably coupled to an electronic suppository egress module 102 and includes circuitry configured to activate responsive to wireless receipt of one or more egress commands from the electronic suppository egress module 102. In an embodiment, the defecation detection module 128 includes circuitry configured to activate responsive to wireless receipt of one or more egress commands from a remote enterprise, a remote user controller, a network, or the like. In an embodiment, the defecation detection module 128 includes circuitry configured to activate responsive to wireless receipt of one or more egress commands from an electronic suppository egress module 102.

In an embodiment, the system 100 includes an electronic suppository 101 having one or more sensors 154. Non-limiting examples of sensors 154 include biosensors, detectors, refractive index detectors, blood volume pulse sensors, conductance sensors, electrochemical sensors, fluorescence sensors, force sensors, heat sensors (e.g., thermistors, thermocouples, or the like), high resolution temperature sensors, differential calorimeter sensors, optical sensors, goniometry sensors, potentiometer sensors, resistance sensors, respiration sensors, sound sensors (e.g., ultrasound), Surface Plasmon Band Gap sensor (SPRBG), physiological sensors, surface plasmon sensors, or the like. Further non-limiting examples of sensors 154 include affinity sensors, bioprobes, biostatistics sensors, enzymatic sensors, in-situ sensors (e.g., in-situ chemical sensor), ion sensors, light sensors (e.g., visible, infrared, or the like), microbiological sensors, microhotplate sensors, micron-scale moisture sensors, nanosensors, optical chemical sensors, single particle sensors, or the like. Further non-limiting examples of sensors include chemical sensors, cavitand-based supramolecular sensors, deoxyribonucleic acid sensors (e.g., electrochemical DNA sensors, or the like), supramolecular sensors, or the like.

Further non-limiting examples of sensors 154 include accelerometers, gyroscopes, position sensors, cameras, radiofrequency sensors, three-dimensional sensors (e.g., 3-D sensors operable to capture information about the shape of a structural features of an intestinal tract, or the like), or the like.

### PROPHETIC EXAMPLES

### Example 1

### A Mobile Electronic Suppository System Operable to Collect Microbial Samples from a Gastrointestinal (GI) Tract and to Map their Locations

In an embodiment, an electronic suppository 101 is used to map, register, identify, quantify, qualify, etc., microbes present in an individual's digestive tract. The system 100 includes an electronic suppository 101 capable of movement in the intestine with sensors 154, that, for example, provides measurand use to determine its location, and pumps and reservoirs to collect samples from intestinal fluids and tissues. In an embodiment, samples are stored in separate reservoirs in a device and information about the samples is transmitted to an external enterprise for storage and analysis. In an embodiment, the electronic suppository 101 includes an egress system operable to expel the electronic suppository 101 from the intestine; and the sample reservoirs are configured to allow recovery of the samples for analysis. In an embodiment, after egress and recovery the electronic suppository 101 can be sterilized and reinserted in the user.

In an embodiment, an electronic suppository 101 is constructed with legs, circuitry, and a power source which allows it to move through the user's intestine. A plastic and metal device with a central core and approximately 3 or more legs which permit travel through the intestine by alternately bracing against the intestinal wall and extending in the direction of travel. For example, a device with jointed legs that is mobile in tubes and channels containing bends and obstructions is described (see e.g., U.S. Pat. No. 5,574,347 issued to Neubauer on Nov. 12, 1996 which is incorporated herein by reference). The articulated legs may be moved by leg controls that include circuitry and motors to actuate the legs in response to sensors on the device or commands from an external operator. In an embodiment, sensors 154 such as pressure sensors, proximity sensors, ultrasound sensors, image sensors, video cameras, etc., may be incorporated in the body of the electronic suppository 101 to locate and guide the device and to signal the leg controls and actuate the leg motors. For example, pressure sensors may detect collision with the intestinal wall indicating a turn in the intestine and signal the leg controls to actuate bracing by one leg and extension of other legs to navigate around the bend in the intestine. Leg controls responsive to sensor signals or other inputs are described (see e.g., U.S. Pat. No. 5,574,347, Ibid.) and motion control circuitry connecting sensors and propulsion mechanisms 209 are described (see e.g., U.S. Patent Publication No. 2007/0225633 by Ferren et al. published on Sep. 27, 2007 which is incorporated herein by reference). In addition, the electronic suppository 101 may be constructed with accelerometers to report the proper acceleration of the electronic suppository 101 through the intestine and the distance traveled which may be used to determine the location of the device and to target a specific anatomic location in the intestine, for example, the jejunum segment in the small intestine.

In an embodiment, the electronic suppository 101 is constructed with sensors to determine its location within the intestine. In an embodiment, information from accelerometers (see above) and sensors may be used to determine that the electronic suppository 101 has traveled approximately 4.0 feet in the large intestine after insertion at the anus which suggests it may have arrived at the cecum or right colon. To confirm the anatomic location of the device, radio telemetric pH sensors are constructed on the electronic suppository 101 to measure and report pH values of the intestinal fluid encountered at selected distances (e.g., 4.0 feet) or selected time points of travel. Radio telemetric pH sensors may be used to measure characteristic pH values in the proximal small bowel (pH 5.9-6.8), distal small bowel (pH 7.4-7.6), cecum/right colon (pH 5.7-6.8) and left colon/rectum (pH 6.1-7.2) (see e.g., Nugent et al., Gut 48: 571-577, 2001 which is incorporated herein by reference). For example an electronic suppository 101 with pH sensors that travels 4.0 feet up the intestine may measure and report a pH between 5.7 and 6.8 corresponding to a location in the cecum/right colon. Further confirmation of the anatomic location of the electronic suppository 101 may be obtained by imaging and processing of radio frequency (RF) signals.

In an embodiment, the electronic suppository 101 is constructed with an imaging system on the device to image the intestine and to help determine the anatomic location of the device. In an embodiment, the body of the electronic suppository 101 is equipped with a miniature video camera, a light source, and a transceiver to send images to an external recording device. For example a miniature imaging system in an ingestible capsule is described (see e.g., U.S. Patent Publication No. 2009/0318766 by Rabinovitz et al. published on Dec. 24, 2009 which is incorporated herein by reference). Computer software and algorithms to analyze transmitted images are available from Given Imaging, Inc., Culver City, Calif. In addition methods to accurately locate the electronic suppository 101 in the intestinal tract by signal processing of RF emissions are described (see e.g., Pourhomayoun et al., Proceedings of the IEEE International Conference of Engineering in Medicine and Biology San Diego, August 2012 which is incorporated herein by reference). In an embodiment, the location of the electronic suppository 101 is determined at the time each microbial sample is collected and the locations are transmitted to an external computer and used to construct a map locating each microbial sample in the intestine and referencing each sample to a storage reservoir in the electronic suppository 101.

In an embodiment, the electronic suppository 101 includes multiple sample collection ports which are controlled by integrated circuitry in the device to collect microbial samples from multiple sites in the intestine, and to store the samples in separate reservoirs in the device. In an embodiment, sample collectors may be constructed using micro-electro-mechanical manufacturing methods. For example, sample collectors may be made on a microchip with multiple tubular ports connected to pumps via valves which are controlled by circuitry on the microchip. In an embodiment, tubular ports may be activated to aspirate liquid and solid samples from the intestinal lumen, biofilms, mucosa and epithelial surfaces of the intestine. In an embodiment, the tubular ports connect to designated sample reservoirs where the microbial samples are stored until they are analyzed. Microchips with sample collectors, sample reservoirs and miniature pumps are described (see e.g., U.S. Pat. No. 8,000,784 issued to Ferren et al. on Aug. 16, 2011; which is incorporated herein by reference). In an embodiment, sample collections are initiated at multiple sites in the intestine by circuitry that may be pre-programmed based on defined distances traveled by the electronic suppository 101 or by anatomic locations determined by the electronic suppository system 100. For example data on the distance traveled, pH, and RF signals may be combined and processed to initiate sample collections in the lumen of the duodenum, jejunum, ileum, and colon. In an embodiment, data from the sensors 154 and video camera on the electronic suppository 101 may be used to initiate sample collection in the lumen, mucus layer, and epithelial surfaces at each intestinal site.

In an embodiment, during operation, once sampling is completed the electronic suppository 101 is recovered by activating an egress system and capturing the device in a toilet. In an embodiment, the egress system includes the legs, motors and motion controls (see above) which may be controlled by RF signals or audible commands to initiate movement of the electronic suppository 101 towards the anus. The electronic suppository 101 may change shape to facilitate movement through constricted regions of the intestine such as the ileocecal valve. A tube crawler which changes shape to navigate obstructions is described (see e.g., U.S. Pat. No. 5,574,347, Ibid.). Also the device may dispense lubricants and laxatives to promote movement of the electronic suppository 101 through the intestine. Following excretion of the electronic suppository 101 into a toilet it is captured by a magnet installed in the toilet. Magnetic force retains the electronic suppository 101 and allows flushing the toilet prior to recovery of the device for sample analysis.

In an embodiment, the samples collected by the electronic suppository 101 may be analyzed by DNA sequence analysis to identify the microbial species present in each sample. In an embodiment, microbial samples are recovered from the reservoirs of the device by activating pumps which are controlled by circuitry on the electronic suppository 101 responsive to external RF signals. Microbial samples approximately 50-100 microliters in volume are processed to obtain microbial DNA. Polymerase chain reaction (PCR) is used to amplify 16s ribosomal RNA (rRNA) genes, and the 16s rRNA genes are sequenced using pyrosequencing (DNA sequencing machines, reagents and protocols for pyrosequencing are available from 454 Life Sciences, Branford, Conn.). Computation methods and algorithms to identify bacterial species by comparison of 16s rRNA gene sequences are employed. For example methods and algorithms to determine intestinal microbiomes based on DNA sequencing are known (see e.g., U.S. Patent Publication No. 2010/0172874 by Tumbaugh et al. published on Jul. 8, 2010 and Cho et al. Nature Reviews Genetics 13: 260-270, 2012 which are incorporated herein by reference).

In an embodiment, the spatial distribution of bacterial phyla and species within an individual's intestine may be compared to distributions determined for healthy controls and/or previous distributions for an individual. For example, in healthy volunteers the small intestine may be enriched for Bacilli, Actinobacteria, Streptococcaceae, Actinomycinaeae and Corynebacteriaceae while the large intestine may be predominately occupied by members of the Bacteriodetes and Firmicutes phyla (see e.g., Sekirov, et al., Physiol. Rev. 90: 859-904, 2010 which is incorporated herein by reference). Also microbes sampled from the mucosal layer and epithelial surface of the small intestine may be mainly three microbial species, Clostridium, Lactobacillus and Enterococcus, but microbes collected in the lumen of the small intestine may include multiple bacterial species including Bacteroides, Bifidobacterium, Streptococcus, Enterobacteriacea, Enterococcus, Clostridium, Lactobacillus and Ruminococcus (see e.g., Sekirov, Ibid.). Furthermore serial sampling and determination of the spatial distribution and composition of microbes in an individual's intestine may indicate microbial imbalances associated with disease. For example increased numbers of Enterobacteriaceae and decreased numbers of Faecalibacterium prausnitzii in the intestine may be associated with inflammatory bowel disease (see e.g., Sekirov, et al., Ibid.). The electronic suppository 101 may be used repeatedly to create a temporal and spatial record of an individual's microbiome which is valuable to healthcare providers and the patient.

In an embodiment, technologies and methodologies are provided for mapping intestinal microbial flora, collecting intestinal microbial flora samples, collecting fluid samples from intestinal mucosa, registering locations of microbial flora, or the like. For example, in an embodiment, the system 100 includes a flora-sampling module 130 having circuitry configured to actuate the collection of at least one intestinal microbial flora sample via a plurality of collection ports 134.

In an embodiment, the flora-sampling module 130 includes circuitry configured to actuate the collection of at least one intestinal microbial flora sample responsive to a target schedule. For example, in an embodiment, the flora-sampling module 130 includes circuitry configured to actuate the collection of at least one intestinal microbial flora sample responsive to time-since-meal information. In an embodiment, the flora-sampling module 130 includes circuitry configured to actuate the collection of at least one intestinal microbial flora sample responsive to one or more inputs indicative of an onset of a defecation event. In an embodiment, the flora-sampling module 130 includes circuitry configured to actuate the collection of at least one intestinal microbial flora sample responsive to one or more inputs indicative of defecation process in progress. In an embodiment, the flora-sampling module 130 includes circuitry configured to actuate the collection of at least one intestinal microbial flora sample responsive to a telemetric input. In an embodiment, the flora-sampling module 130 includes circuitry configured to actuate the collection of at least one intestinal microbial flora sample responsive to an electronic suppository external capture protocol.

In an embodiment, the system 100 includes a flora-sampling module 130 having circuitry configured to actuate the collection of one or more intestinal microbial flora samples, fluid samples from intestinal mucosa, or the like. For example, in an embodiment, the flora-sampling module 130 includes circuitry configured to actuate the collection of the one or more intestinal microbial flora samples responsive to target schedule information. In an embodiment, the flora-sampling module 130 includes circuitry configured to actuate the collection of one or more fluid samples from intestinal mucosa responsive to target schedule information

In an embodiment, the flora-sampling module 130 includes circuitry configured to actuate the collection of the one or more intestinal microbial flora samples responsive to time-since-meal information. In an embodiment, the flora-sampling module 130 includes circuitry operably coupled to a collection-site scraper assembly, and configured to actuate the scraping of a collection site of prior to collection of the one or more intestinal microbial flora samples.

In an embodiment, the system 100 includes a mucosal collection assembly configured to penetrate the mucosal and to collect at least one intestinal microbial flora sample. For example, in an embodiment, the flora-sampling module 130 includes circuitry operably coupled to a biopsy assembly 302 configured to obtain mucosal tissue biopsies. In an embodiment, the flora-sampling module 130 includes circuitry operably coupled to a biopsy assembly 302 configured to obtain microbial flora biopsies. In an embodiment, the flora-sampling module 130 includes circuitry configured to actuate a probe assembly operable to collect the at least one intestinal microbial flora sample from a target mucosal depth.

In an embodiment, the biopsy assembly 302 includes one or more articulated structure elements. In an embodiment, the biopsy assembly 302 includes one or more telescopic structural elements. In an embodiment, the biopsy assembly 302 includes one or more retractable structural elements. In an embodiment, the biopsy assembly 302 includes one or more extendible structural elements.

In an embodiment, the biopsy assembly 302 includes one or more articulated biopsy probes. In an embodiment, the biopsy assembly 302 includes one or more telescopic biopsy probes. In an embodiment, the biopsy assembly 302 includes one or more retractable biopsy probes. In an embodiment, the biopsy assembly 302 includes one or more extendible biopsy probes.

In an embodiment, the biopsy assembly 302 includes one or more collection probes. In an embodiment, the biopsy assembly 302 includes one or more articulated structures configured to scrape away matter form a biological surface.

In an embodiment, the system 100 includes one or more collection reservoirs 136 configured to receive and store the at least one intestinal microbial flora sample. For example, in an embodiment, the system 100 includes one or more environment-controlled collection reservoirs configured to receive and store the at least one intestinal microbial flora sample. In an embodiment, the system 100 includes one or more temperature-controlled collection reservoirs 136 configured to receive and store the at least one intestinal microbial flora sample. In an embodiment, the system 100 includes one or more refrigerated collection reservoirs 136 configured to receive and store the at least one intestinal microbial flora sample.

In an embodiment, the system 100 includes an electronic suppository egress module 102 including circuitry configured to actuate an egress protocol associated with egress of an electronic suppository 101 from an intestinal tract. In an embodiment, the system 100 includes an electronic suppository egress module 102 operably coupled to a lubricant supply module 118 and is configured to deliver at least one lubricating material to an outer surface on an electronic suppository 101.

In an embodiment, the electronic suppository egress module 102 includes circuitry configured to actuate delivery of at least one lubricating material responsive to activation of an egress protocol associated with egress of an electronic suppository 101 from an intestinal tract. In an embodiment, the system 100 includes an electronic suppository egress module 102 operably coupled to a laxative supply module 126 configured to deliver at least one laxative composition to an outer surface on an electronic suppository 101. In an embodiment, the electronic suppository egress module 102 includes circuitry configured to actuate delivery of at least one laxative composition responsive to activation of an egress protocol associated with egress of an electronic suppository 101 from an intestinal tract. In an embodiment, the system 100 includes an electronic suppository egress module 102 having circuitry configured to actuate a change in a physical dimension of an electronic suppository 101 to facilitate egress of the electronic suppository from an intestinal tract.

In an embodiment, the system 100 includes a communication module 138 having circuitry 140 configured to negotiate user-specific microbial flora information based on at least one cryptographic protocol, encryption protocol, or decryption protocol. In an embodiment, the system 100 includes a communication module 138 operably coupled to a speech recognition module 139 that causes the communication module associated with an electronic suppository 101 to change between a transmit states and a receive state responsive to one or more audio inputs. In an embodiment, the system 100 includes a communication module 138 having one or more receivers 104, transmitters 106, transceivers 108, or the like operable to communicate with a remote enterprise and to receive information (e.g., suppository egress protocol information, user-specific flora information, target seeding locations information, etc.) from the remote enterprise.

In an embodiment, the system 100 includes a communication module 138 having one or more receivers 104, transmitters 106, transceivers 108, or the like operable to report intent to egress to, for example, a remote network. In an embodiment, the system 100 includes a communication module 138 having one or more receivers 104, transmitters 106, transceivers 108, or the like operable to report activation of egress. In an embodiment, the system 100 includes a communication module 138 having one or more receivers 104, transmitters 106, transceivers 108, or the like operable to report an onset of an egress event. In an embodiment, the system 100 includes a communication module 138 having one or more receivers 104, transmitters 106, transceivers 108, or the like operable report travel information during an egress event. In an embodiment, the system 100 includes a communication module 138 having one or more receivers 104, transmitters 106, transceivers 108, or the like operable to report time-till-exit information.

In an embodiment, the system 100 includes a power source 142. In an embodiment, the system 100 includes a reusable power source 142. Non-limiting examples of power source 142 include one or more button cells, chemical battery cells, a fuel cell, secondary cells, lithium ion cells, micro-electric patches, nickel metal hydride cells, silver-zinc cells, capacitors, super-capacitors, thin film secondary cells, ultra-capacitors, zinc-air cells, and the like. Further non-limiting examples of power sources 142 include one or more generators (e.g., electrical generators, thermo energy-to-electrical energy generators, mechanical-energy-to-electrical energy generators, micro-generators, nano-generators, or the like) such as, for example, thermoelectric generators, piezoelectric generators, electromechanical generators, biomechanical-energy harvesting generators, and the like. In an embodiment, the electronic suppository 101 includes one or more generators configured to harvest mechanical energy from for example, ultrasonic waves, mechanical vibration, blood flow, and the like. In an embodiment, the electronic suppository 101 includes one or more power receivers configured to receive power from an in vivo or ex vivo power source.

In an embodiment, the electronic suppository 101 includes one or more biological-subject-powered generators configured to harvest thermal energy generated by a biological subject. For example, in an embodiment, the electronic suppository 101 includes one or more thermoelectric generators configured to harvest heat dissipated by the biological subject. In an embodiment, the electronic suppository 101 includes one or more biological-subject-powered generators configured to harvest energy generated by any physical motion or movement (e.g., walking,) by biological subject. In an embodiment, the electronic suppository 101 includes one or more biological-subject-powered generators configured to harvest energy generated by the movement of a fluid (e.g., biological fluid, blood, cerebrospinal fluid, etc.) within the biological subject.

In an embodiment, the electronic suppository 101 includes at least one of a thermoelectric generator, a piezoelectric generator, a microelectromechanical system generator, or a biomechanical-energy harvesting generator. In an embodiment, the system 100 includes a transcutaneous energy transfer system. In an embodiment, the system 100 includes an electronic suppository 101 electromagnetically, magnetically, ultrasonically, optically, inductively, electrically, or capacitively coupled to a transcutaneous energy transfer system. In an embodiment, the electronic suppository 101 includes a power source 142 including at least one battery. In an embodiment, the electronic suppository 101 includes a power source 142 wired or wireless coupled to an external source. In an embodiment, the electronic suppository 101 includes a power source 142 including at least one of a thermoelectric generator, a piezoelectric generator, a microelectromechanical system generator, or a biomechanical-energy harvesting generator. In an embodiment, the electronic suppository 101 includes a power source 142 electromagnetically, magnetically, ultrasonically, optically, inductively, electrically, or capacitively coupled to a power supply.

In an embodiment, the system 100 includes a flora-mapping module 152. In an embodiment, the system 100 includes a flora-mapping module 152 including circuitry configured to generate flora collection information associated with at least one intestinal microbial flora sample. In an embodiment, the flora-mapping module 152 includes circuitry configured to generate travel path information associated with the collection of the intestinal microbial flora sample. For example, in an embodiment, the flora-mapping module 152 includes one or more sensors 154 that acquire intestinal travel path markings information. In an embodiment, the flora-mapping module 152 includes one or more travel route sensors configured to detect a travel distance along intestinal travel route. In an embodiment, the flora-mapping module 152 includes one or more travel route sensors configured to detect a rate of travel.

In an embodiment, the flora-mapping module 152 includes circuitry configured to register one or more target regions within an intestinal tract. For example, in an embodiment, the flora-mapping module 152 includes one or more computing devices that compare a detected thermograph from at least one sensor 154 associated with one or more landmark features of an intestinal region to reference thermograph information (e.g., thermographic data, thermographic images, etc.) and generates registration information (e.g., target registration information, user-specific target registration information, user-specific intestinal registration information, or the like, based on the comparison. In an embodiment, the registration information includes one or more of a location coordinate (e.g., a seeding location coordinate, a collection site location coordinate, a laxative delivery location coordinate, etc.), a region dimension, a region depth, or a region beam axis direction. In an embodiment, the treatment registration information includes anatomical target identification information, anatomical target location information, anatomical target shape information, anatomical target dimension information, anatomical target distribution information, or point cloud information.

In an embodiment, the flora-mapping module 152 includes circuitry configured to initiate a discovery protocol that allows the flora-mapping module 152 and a remote enterprise to identify each other and negotiate one or more pre-shared keys. In an embodiment, the flora-mapping module 152 includes at least one of a receiver 104, transmitter 106, and a transceiver 108 operable to actuate a discovery protocol that allows an electronic suppository 101 and a remote enterprise to identify each other and negotiate information.

In an embodiment, the flora-mapping module 152 includes one or more sensors 154 operable to detect (e.g., assess, calculate, evaluate, determine, gauge, measure, monitor, qualify, quantify, resolve, sense, or the like) at least one characteristic (e.g., a spectral characteristic, a spectral signature, a physical quantity, an environmental attribute, a physiologic characteristic, a response associated with a focal volume interrogated by an electromagnetic energy stimulus, or the like) associated with a biological sample (e.g., bacteria, microbes, single-cell eukaryotes viruses, tissue, biological fluids, biomarkers, a microbial flora, or the like). In an embodiment, the flora-mapping module 152 includes circuitry configured to acquire intestinal anatomical features information. For example, in an embodiment, the flora-mapping module 152 includes one or more sensors 154 configured to detect (e.g., optically detect, acoustically detect, thermally detect, energetically detect, spectroscopically detect, or the like) one or more intestinal anatomical features within an intestinal tract.

In an embodiment, the system 100 includes an electronic suppository 101 operable to register a target location relative to a portion of an intestinal tract. For example, during operation, the flora-mapping module 152 maps (e.g., spatially aligns, registers, projects, correlates, etc.) the geographical location of electronic suppository 101 relative to the geographical location of the intestinal tract. In an embodiment, the flora-mapping module 152 is configured to generate one or more egress control commands for actuating an egress of an electronic suppository 101 responsive to registration information. In an embodiment, the flora-mapping module 152 registers a plurality of objects by mapping coordinates from one object to corresponding points in another object. In an embodiment, the flora-mapping module 152 registers objects (e.g., travel path locations, target and reference objects, targets and focal regions, images, etc.) using one or more transformations.

In an embodiment, the flora-mapping module 152 registers objects (e.g., travel path locations, target and reference objects, targets and focal regions, images, etc.) using one or more registration techniques or methodologies. Non-limiting examples of registration techniques or methodologies include deformable registration, landmark-based registration, or rigid registration. See e.g., Paquin et al., Multiscale Image Registration, Mathematical Biosciences and Engineering, Vol. 3:2 (2006); see also Paquin, Dana, PhD, Multiscale Methods for Image Registration, Ph.D. dissertation, Stanford University (2007); Zitova et al., Image Registration Methods: a Survey, Image and Vision Computing (21) pp. 977-1000 (2003); each of which is incorporated herein by reference. In an embodiment, registration includes techniques or methodologies for spatially aligning images taken using different imaging modalities, taken at different times, or that vary in perspective. Further non-limiting examples of registration techniques or methodologies include deformable multiscale registration, hybrid multiscale landmark registration, multiscale image registration, or rigid multiscale registration. In an embodiment, registration includes one or more of feature detection, feature identification, feature matching, or transform modeling. In an embodiment, registration includes mapping features of a first object with the features of a second object. In an embodiment, registration includes determining a point-by-point correspondence between two objects, regions, or the like. In an embodiment, registration includes determining a point-by-point correspondence between an object and a location. For example, in an embodiment, registration includes determining a point-by-point correspondence between an object and a region of an intestinal tract.

In an embodiment, the flora-mapping module 152 is configured to register the electronic suppository 101 relative to a portion of an intestinal tract and to generate registration information. In an embodiment, the flora-mapping module 152 includes a registration module operable to register one or more microbial flora collection location targets responsive to a collection event. In an embodiment, the flora-mapping module 152 includes circuitry configured to active registration of one or more microbial flora collection location targets. In an embodiment, the flora-mapping module 152 includes circuitry configured to generate the flora collection information responsive to detection of one or more spectral components associated with an intestinal tract anatomy. In an embodiment, the flora-mapping module 152 includes circuitry configured to generate next-in-time microbial flora collection location information.

In an embodiment, the flora-mapping module 152 includes circuitry configured to identify a target collection site within an intestinal tract based on a detected measurand associated with an intestinal anatomical target location. In an embodiment, the flora-mapping module 152 includes circuitry configured to identify a target collection site within an intestinal tract responsive to a collection event. In an embodiment, the flora-mapping module 152 includes circuitry configured to identify a target collection site within an intestinal tract based on one or more detected anatomical features. In an embodiment, the flora-mapping module 152 includes circuitry configured to generate flora collection information based on a comparison between at least one datum associated with a detected anatomical feature of an intestinal tract and reference intestinal tract information.

In an embodiment, the system 100 includes one or more electronic suppositories 101. The electronic suppository 101 can take a variety of shapes, configurations, and geometric forms including regular or irregular forms and can have a cross-section of substantially any shape including, for example, circular, triangular, square, rectangular, polygonal, regular or irregular shapes, or the like, as well as other symmetrical and asymmetrical shapes, or combinations thereof.

In an embodiment, an electronic suppository 101 includes a housing structure 204. In an embodiment, an electronic suppository 101 includes a housing structure 204 having a plurality of segments. In an embodiment, one or more portions of the housing structure 204 take a variety of shapes, configurations, and geometric forms including regular or irregular forms. For example, in an embodiment, one or more portions of the housing structure 204 form part of a substantially cylindrical geometric structure (e.g., a tubular structure) having an inner surface defining one or more compartments. In an embodiment, the housing structure 204 forms part of a substantially cylindrical geometric structure having a cross-section of substantially any shape including, for example, circular, triangular, square, rectangular, polygonal, regular or irregular shapes, or the like, as well as other symmetrical and asymmetrical shapes, or combinations thereof.

In an embodiment, the electronic suppository 101 includes a multi-segment housing structure 204 having multiple structures physically coupled to each other. For example, in an embodiment, the housing structure 204 includes a plurality of connected segments 204a, 204b, 204c, 204d, etc. In an embodiment, the electronic suppository 101 includes a housing structure 204 having a plurality of segments 204a, 204b, 204c, 204d, etc., physically coupled along a longitudinal length. In an embodiment, the electronic suppository 101 includes a housing structure 204 having a plurality of segments 204a, 204b, 204c, 204d, etc., in fluid communication. In an embodiment, the electronic suppository 101 includes a housing structure 204 having a plurality of segment 204a, 204b, 204c, 204d, etc., connected via separate components. In an embodiment, the electronic suppository 101 includes a housing structure 204 configured as a monolithically structure.

In an embodiment, the electronic suppository 101 includes a housing structure 204 configured as an integrally formed component assembly. In an embodiment, the housing structure 204 includes a plurality of segments 204a, 204b, 204c, 204d, etc., in fluid communication via a plurality of selectively controllable fluid-flow passageways. In an embodiment, the housing structure 204 includes a plurality of segments configured to store one or more solid, liquid, homogenous, heterogeneous, etc., microbial flora samples. In an embodiment, the electronic suppository 101 includes a housing structure 204 having a plurality of reusable tubular structures.

In an embodiment, an electronic suppository 101 includes a housing structure 204 having at least one payload module 210 and at least one reusable power module 212. In an embodiment, the reusable power module 212 includes one or more power sources 142. In an embodiment, the reusable power module 212 includes a communication module 214 including circuitry configured at least one of a receiver 104, transmitter 106, and a transceiver 108 operable to communicate with a remote enterprise and to receive control command information from the remote enterprise.

In an embodiment, an electronic suppository 101 includes payload module 210 including a plurality of collection ports 134 arranged to provide fluidic communication between an interior environment of the housing structure and an exterior environment, and configured to acquire an intestinal microbial flora sample. In an embodiment, an electronic suppository 101 includes payload module 210 including at least one reservoir operably coupled via at least one flow path to one or more of the plurality of collection ports and configured to receive and store an intestinal microbial flora sample. In an embodiment, the payload module 210 includes circuitry configured to actuate the collection of at least one intestinal microbial flora sample via at least one of the plurality of collection ports. In an embodiment, the payload module 210 includes circuitry configured to generate flora collection information associated with at least one intestinal microbial flora sample.

In an embodiment, an electronic suppository 101 includes a flora-sampling module 130 received in the housing structure 204. In an embodiment, the flora-sampling module 130 includes a plurality of collection ports 134 arranged to provide fluidic communication between an interior environment of the housing structure 204 and an exterior environment, and configured to acquire an intestinal microbial flora sample. In an embodiment, the flora-sampling module 130 includes at least one collection reservoir 136 operably coupled via at least one flow path 206 to one or more of the plurality of collection ports 134 and configured to receive and store an intestinal microbial flora sample. In an embodiment, the flora-sampling module 130 includes circuitry configured to actuate the collection of at least one intestinal microbial flora sample via at least one of the plurality of collection ports 134. In an embodiment, the flora-sampling module 130 includes circuitry configured to controlled an internal temperature of the at least one reservoir. In an embodiment, the flora-sampling module 130 includes circuitry configured to control a refrigeration process associated with a sample in the at least one reservoir.

In an embodiment, an electronic suppository 101 includes a flora-mapping module 152 including circuitry configured to generate flora collection information associated with at least one intestinal microbial flora sample. In an embodiment, the flora-mapping module 152 includes one or more sensors 154 operable to detect an environmental condition. In an embodiment, an electronic suppository 101 includes a flora-mapping module 152 including at least one of a receiver 104, transmitter 106, and a transceiver 108 operable to communicate microbial flora information.

In an embodiment, an electronic suppository 101 includes a suppository navigation module 208 including circuitry configured to actuate advancement of the electronic suppository along an interior of an intestinal tract. In an embodiment, an electronic suppository 101 includes a suppository navigation module 208 operably coupled to a propulsion mechanism 209, and configured to cause the propulsion mechanism 209 to advance an electronic suppository 101 along an interior of an intestinal tract. For example, during operation, in an embodiment, intestinal tract wall engagement structures 213 forming part of the propulsion mechanism 209, and operable to alternate engagement and disengagement by extending or retracting, physically contact a portion of an intestinal wall. In an embodiment, an inchworm motor device 211 operably coupled to the engagement structures 213 enables the electronic suppository 101 to move along an intestinal tract by alternate engagement and disengagement of the intestinal wall by at least a first and second engagement structures 213. This alternate engagement and disengagement of the intestinal wall by at least a first and second engagement structures 213 may produce inchworm-type propulsion of the electronic suppository 101 along an interior of an intestinal tract.

Further non-limiting examples of propulsion mechanisms 209 are described in U.S. Patent Publication No. 2007/0225633 (by Ferren et al. published on Sep. 27, 2007; which is incorporated herein by reference). In an embodiment, the electronic suppository 101 includes a propulsion mechanism 209 having structural elements configured to physically contact two or more portion of an intestinal wall, to frictionally couple the ingestible flora-sampling device to an intestinal wall, and to move the electronic suppository 101 along an intestinal wall. For example, the electronic suppository 101 includes a propulsion mechanism 209 having structural elements that adhere to the an intestinal wall, via a suction (negative pressure) generating mechanism. (See e.g., U.S. Patent Publication No. 2007/0225633).

In an embodiment, the electronic suppository 101 includes a propulsion mechanism 209 having extendable mechanism having structural elements having one or more mechanical legs configured to contact two or more portion of an intestinal wall. In an embodiment, the propulsion mechanism 209 includes structural elements configured to frictionally couple the electronic suppository 101 to an intestinal wall during collection of at least one intestinal microbial flora sample. In an embodiment, the structural elements include one or more inflatable elements. In an embodiment, the structural elements include one or more bimorphs (e.g., bimorph actuators, bimorph cantilevers, piezoelectric bimorphs, etc.). In an embodiment, the electronic suppository 101 includes a propulsion mechanism 209 having structural elements 211 configured to physically contact two or more portion of an intestinal wall, to frictionally couple the ingestible flora-sampling device to an intestinal wall, and to move the electronic suppository 101 along an intestinal wall. For example, in an embodiment, the propulsion mechanism 209 includes an inchworm motor device 213 that enables the electronic suppository 101 to move along an intestinal tract.

Referring to FIG. 2, in an embodiment, the system 100 includes a virtual object generation module 252. In an embodiment, the electronic suppository 101 includes a virtual object generation module 252. For example, in an embodiment, the electronic suppository 101 includes a virtual object generation module 252 operably coupled to the flora-mapping module 152. In an embodiment, during operation, the virtual object generation module 252 is configured to generate a virtual representation 254 of at least one of a locality of the electronic suppository 101 within an intestinal tract, a locality of a microbial flora collection site, a locality of a registration landmark, etc., within a physical space on a remote virtual display 256. In an embodiment, during operation, an onboard virtual object generation module 252 is configured to generate a virtual representation indicative of flora collection information on a remote display. In an embodiment, during operation, the virtual object generation module 252 is configured to generate a virtual representation indicative of flora mapping information. In an embodiment, during operation, the virtual object generation module 252 is configured to generate a virtual representation indicative of flora seeding information.

In an embodiment, the flora-mapping module 152 is configured to track at least a portion of the electronic suppository 101 within an intestinal tract and to update a virtual object 258 in a virtual space corresponding to the physical location of at least one of the electronic suppository 101. In an embodiment, the electronic suppository 101 includes a virtual object generation module 252 operably coupled to an electronic suppository navigation module 208 and configured to generate a virtual representation 260 of the one or more electronic suppository 101 navigation control commands on a virtual display.

In an embodiment, the electronic suppository 101 includes a virtual object generation module 252 operably coupled to an intestinal-microbial-flora-seeding module and configured to generate a virtual representation 262 corresponding to the physical location of a seeding event on a virtual display. In an embodiment, the electronic suppository 101 includes a virtual object generation module 252 operably coupled to an intestinal-microbial-flora-seeding module and configured to generate a virtual representation 262 corresponding to the physical location of the electronic suppository 101 within an intestinal tract, on a virtual display. In an embodiment, the electronic suppository 101 includes a virtual object generation module 252 configured to generate a virtual representation 264 corresponding to registration of the electronic suppository 101 and a target location on a virtual display. In an embodiment, the electronic suppository 101 is configured to image one or more physical movements of the electronic suppository 101 within the intestinal tract responsive to sensor 154 information and to update a virtual object 260 in a virtual space corresponding to the one or more physical movements of the electronic suppository 101 within the intestinal tract.

In an embodiment, technologies and methodologies are provided for seeding an intestinal tract with microbial flora. Referring to FIG. 2, in an embodiment, an intestinal-microbial-flora-seeding suppository 101a includes a housing structure 204, a plurality of microbial flora reservoirs 272, a plurality of selectively actuatable delivery ports 274 connected to the plurality of reservoirs 272, and a microbial flora delivery module 276. In an embodiment, the microbial flora delivery module 276 includes circuitry configured to actuate the delivery of an intestinal microbial flora composition from one or more the plurality of microbial flora reservoirs 272 to an exterior environment of the housing structure 204 responsive to user-specific microbial flora seeding information. For example, in an embodiment, a plurality of selectively actuatable delivery ports 274 is operably coupled to one or more of the plurality of microbial flora reservoirs 272 to an exterior environment of the housing structure 204.

In an embodiment, the microbial flora delivery module 276 controls delivery of an intestinal microbial flora composition based on a user-specific protocol stored in memory 110, or wirelessly received from a remote enterprise, a remote controller, a network, a remote module, a remote device, or the like. For example, in an embodiment, during operation, the microbial flora delivery module 276 controls a seeding process by controlling the plurality of selectively actuatable delivery ports 274 between OPEN or CLOSED states to cause delivery of an intestinal microbial flora composition stored in one more of the plurality of microbial flora reservoirs 272 to an exterior environment responsive to user-specific microbial flora seeding information stored in one or more memories 110. In an embodiment, the microbial flora delivery module 276 includes circuitry operable to control OPEN or CLOSED states of one or more valves along a flow path between at least one reservoir and an exterior environment of an electronic suppository 101.

In an embodiment, the plurality of selectively actuatable delivery ports 274 is effective to deliver an intestinal microbial flora composition received in one more of the plurality of microbial flora reservoirs 272 to an exterior environment responsive to target registration information generated by one or more flora-mapping module 152 or the like, and indicative of an alignment of a target region within an intestinal tract with an intestinal microbial flora composition zone associated with one or more of the plurality of selectively actuatable delivery ports 274. For example, in an embodiment, the plurality of selectively actuatable delivery ports 274 is effective to deliver an intestinal microbial flora composition received in one more of the plurality of microbial flora reservoirs 272 to an exterior environment to responsive to a measurand from one or more sensors 154 indicating that an electronic suppository 101 is proximate a target region within an intestinal tract. In an embodiment, the plurality of microbial flora reservoirs 272 includes one or more insulated microbial flora reservoirs.

In an embodiment, the plurality of selectively actuatable delivery ports 274 is effective to deliver an intestinal microbial flora composition received in one more of the plurality of microbial flora reservoirs 272 to an exterior environment responsive to target registration information stored in memory 110, or wirelessly received from a remote enterprise, a remote controller, a network, a remote module, a remote device, or the like.

In an embodiment, the plurality of microbial flora reservoirs 272 includes one or more detachable microbial flora reservoirs. For example, in an embodiment, a user can select from a number of microbial flora reservoirs, each loaded with a different intestinal microbial flora composition. In an embodiment, each microbial flora reservoir 272 is configured to removably attach to a modular structure that is loaded into, or forms part of an intestinal-microbial-flora-seeding suppository 101a. Accordingly, in an embodiment, users can customize the intestinal-microbial-flora-seeding suppository's payload by choosing the number of microbial flora reservoirs 272 and the specific kinds of intestinal microbial flora compositions to be used. For example, in an embodiment, the plurality of microbial flora reservoirs 272 includes at least a first detachable microbial flora reservoir and a second detachable microbial flora reservoir. In an embodiment, the second detachable microbial flora reservoir carries a different microbial flora composition from a microbial flora composition received in the first detachable microbial flora reservoir

In an embodiment, the plurality of microbial flora reservoirs 272 includes one or more environment-controlled microbial flora reservoirs. For example, in an embodiment, the plurality of microbial flora reservoirs 272 includes one or more temperature-controlled microbial flora reservoirs. For example, in an embodiment, an intestinal-microbial-flora-seeding suppository 101a includes at one Micro Electro Mechanical System (MEMS) microcapillary pumped loop-cooling device operable to control the temperature of a microbial flora reservoirs 272. Non-limiting examples of MEMS microcapillary pumped loop-cooling devices are described in U.S. Pat. No. 6,976,527 (issued on Dec. 20, 2005; which is incorporated herein by reference). in an embodiment, an intestinal-microbial-flora-seeding suppository 101a includes one or more conductive traces operable to control the temperature of a microbial flora reservoirs 272 via resistive heating in the presence of an applied current.

In an embodiment, the plurality of microbial flora reservoirs 272 includes one or more refrigerated microbial flora reservoirs. For example, in an embodiment, an intestinal-microbial-flora-seeding suppository 101a includes a micro cryogenic cooling device, including one or more micro-compressors and a refrigerant, operable to cool a sample received in a microbial flora reservoir 272. In an embodiment, an intestinal-microbial-flora-seeding suppository 101a includes one or more Joule-Thompson based micro cryogenic coolers operable to cool a sample received in a microbial flora reservoir 272. Non-limiting examples of micro cryogenic cooling devices are described in, for example, U.S. Patent Pub No. 2008/0178606 (Published on Jul. 31, 2008), and Lewis et al., Demonstration of an Integrated Micro Cryogenic Cooler and Miniature Compressor for Cooling to 200K, Proceedings of the International Mechanical Engineering Congress and Exposition IMECE 2011 (Nov. 14-17, 2011) (each of which is incorporated herein by reference).

In an embodiment, an intestinal-microbial-flora-seeding suppository 101a includes a quantum refrigerator device operable to cool a sample received in a microbial flora reservoir 272. Non-limiting examples of quantum refrigerator devices are described in P. J. Lowell et al., Macroscale refrigeration by nanoscale electron transport. Applied Physics Letters. 102, 082601 (2013) (Published online 26 Feb. 26, 2013. http://dx.doi.org/10.1063/1.4793515); which is incorporated herein by reference).

In an embodiment, an intestinal-microbial-flora-seeding suppository 101a includes a plurality of microbial flora reservoirs forming part of a modular structure. For example, in an embodiment, one or more of the plurality of microbial flora reservoirs forms part of a modular structure. In an embodiment, one or more of the plurality of microbial flora reservoirs forms part of a modular structure detachably coupleable to the housing structure. In an embodiment, the plurality of microbial flora reservoirs forms part of a module configured to detachably couple to the housing structure. In an embodiment, one or more of the plurality of microbial flora reservoirs are detachably coupleable to the module configured to detachably couple to the housing structure.

In an embodiment, the microbial flora delivery module 276 includes circuitry operable to control ON or OFF states of a plurality of selectively actuatable delivery ports. In an embodiment, the microbial flora delivery module 276 includes circuitry operable to control OPEN or CLOSED states of one or more valves along a flow path between at least one reservoir and an exterior environment of an electronic suppository 101. In an embodiment, by controlling, for example, an ON or OFF states of a plurality of selectively actuatable electromechanical delivery ports, or OPEN or CLOSED states of one or more electromechanical valves, the microbial flora delivery module 276 is operable to control at least one parameter associated with a delivery rate, a delivery amount, and a delivery frequency associated with the delivery of the intestinal microbial flora composition. In an embodiment, by controlling, for example, an ON or OFF states of a plurality of selectively actuatable electromechanical delivery ports, the microbial flora delivery module 276 is operable to control one or more parameters associated with a port release rate, a port release amount, and a port release pattern associated with the delivery of the intestinal microbial flora composition from at least one reservoir through one or more ports, valves, flow paths, etc., to an exterior environment of an electronic suppository 101.

In an embodiment, the microbial flora delivery module 276 includes circuitry operable to activate delivery of a microbial flora composition responsive to one or more patient-specific instructions. In an embodiment, the microbial flora delivery module 276 includes circuitry operable to control delivery of the microbial flora composition responsive to a time protocol. In an embodiment, the microbial flora delivery module 276 includes circuitry operable to control delivery of the microbial flora composition responsive to an input indicative of a target location along an intestinal tract.

In an embodiment, the microbial flora delivery module 276 includes one or more sensors 154 and is operable to deliver a composition responsive to a sensed environmental condition. In an embodiment, the microbial flora delivery module 276 includes circuitry operable to activate delivery of the microbial flora composition responsive to a measurand indicative of a target region within an intestinal tract. In an embodiment, the microbial flora delivery module 276 includes circuitry configured to operably couple the electronic suppository system to one or more remote sensors. In an embodiment, the microbial flora delivery module 276 includes circuitry configured to activate delivery of a microbial flora composition responsive to at least one measurand input from the one or more remote sensors 154 indicative of a target region within an intestinal tract. For example, during operation, in an embodiment, the electronic suppository 101 includes circuitry including one or more sensors 154 operable to capture a spectral profile of a region within an intestinal tract. In an embodiment, the electronic suppository 101 further includes circuitry operable to compare the spectral profile to reference spectral information and to determine whether any portion of the spectral profile is indicative of a target region within an intestinal tract.

In an embodiment, the microbial flora delivery module 276 includes circuitry operable to initiate an egress protocol. For example, in an embodiment, the microbial flora delivery module 276 includes one or more reservoirs and is configured to deliver lubricant to an outer surface of the electronic suppository to initiate egress of the electronic suppository 101. In an embodiment, the microbial flora delivery module 276 includes an intestinal tract registration module including circuitry configured to register one or more intestinal tract location with one or more virtual target microbial flora seeding locations. In an embodiment, the microbial flora delivery module 276 includes circuitry configured to generate microbial flora seeding information responsive to a microbial flora delivery event.

Referring to FIG. 3, in an embodiment, an ingestible flora-sampling device 301 includes a flora-sampling module 130 and a flora-mapping module 152. In an embodiment, the ingestible flora-sampling device 301 includes an egress module 102 including circuitry configured to control an egress process associated with egress of the ingestible flora-sampling device 301 from an intestinal tract.

In an embodiment, the flora-sampling module 130 includes circuitry configured to actuate collection of at least one intestinal microbial flora sample via a plurality of collection ports 134. In an embodiment, the flora-sampling module 130 includes circuitry configured to actuate the collection of at least one intestinal microbial flora sample responsive to a target schedule. In an embodiment, the flora-sampling module 130 includes circuitry configured to actuate the collection of a plurality of intestinal microbial flora samples, fluid samples from intestinal mucosa, or the like at one or more time intervals. In an embodiment, the flora-sampling module 130 includes circuitry configured to actuate the collection of a plurality of intestinal microbial flora samples at regular or irregular time intervals.

In an embodiment, the flora-sampling module 130 includes circuitry configured to actuate the collection of a plurality of intestinal microbial flora samples responsive to an indication from one or more sensor 154 of distance traveled. In an embodiment, the flora-sampling module 130 includes circuitry configured to actuate the collection of a plurality of intestinal microbial flora samples responsive to one or more inputs from an inertial sensor indicative of a target orientation of an ingestible flora-sampling device 301. In an embodiment, the flora-sampling module 130 includes circuitry configured to actuate the collection of a plurality of intestinal microbial flora samples responsive to one or more inputs from an inertial sensor indicative of a target angular rate an ingestible flora-sampling device 301. In an embodiment, the flora-sampling module 130 includes circuitry configured to actuate the collection of a plurality of intestinal microbial flora samples responsive to one or more inputs from an inertial sensor indicative of a target linear acceleration an ingestible flora-sampling device 301.

In an embodiment, the flora-sampling module 130 includes circuitry configured to actuate the collection of a plurality of intestinal microbial flora samples, the collection of a plurality of fluid samples from intestinal mucosa, or the like responsive to one or more inputs from an environmental sensor indicative of a target pH. In an embodiment, the flora-sampling module 130 includes circuitry configured to actuate the collection of a plurality of intestinal microbial flora samples responsive to one or more inputs from an environmental sensor indicative of a target pH change.

In an embodiment, the flora-sampling module 130 includes circuitry configured to actuate the collection of a plurality of intestinal microbial flora samples responsive to one or more inputs from a sensor indicative of physical contact with intestinal wall. In an embodiment, the flora-sampling module 130 includes circuitry configured to actuate the collection of a plurality of intestinal microbial flora samples responsive to one or more inputs from a sensor indicative of physical contact with mucosa.

In an embodiment, the flora-sampling module 130 includes circuitry configured to actuate the collection of a plurality of intestinal microbial flora samples responsive to a patient-specific collection protocol. In an embodiment, the flora-sampling module 130 includes one or more reservoirs for storing the at least one intestinal microbial flora sample. In an embodiment, the flora-sampling module 130 includes a plurality of reservoirs for storing a plurality of intestinal microbial flora samples in series or in parallel.

In an embodiment, the flora-sampling module 130 includes a plurality of reservoirs for storing a plurality of intestinal microbial flora samples in series responsive to a target schedule. In an embodiment, the flora-sampling module 130 includes a plurality of reservoirs for storing a plurality of intestinal microbial flora samples responsive to regular or irregular time intervals.

In an embodiment, the flora-sampling module 130 includes a plurality of reservoirs for storing a plurality of intestinal microbial flora samples in series or in parallel responsive to at least one input indicative of a target location.

In an embodiment, the flora-sampling module 130 includes a plurality of selectively controllable reservoirs 136 for storing a plurality of intestinal microbial flora samples. In an embodiment, the flora-sampling module 130 includes a plurality of independently controlled reservoirs 136 for storing a plurality of intestinal microbial flora samples. In an embodiment, the flora-sampling module 130 includes a plurality of reservoirs and circuitry configured to actuate one or more of the plurality of collection ports 134 between an open state and closed state responsive to a measurand indicative of received sample with one or more of the plurality of reservoirs 136.

In an embodiment, the flora-sampling module 130 includes a sample-collection assembly 304 configured to obtain microbial flora samples. In an embodiment, the flora-sampling module 130 includes circuitry operably coupled to a sample-collection assembly 304 configured to obtain microbial flora biopsies. In an embodiment, the sample-collection assembly 304 is operable to collect at least one intestinal microbial flora sample from a target mucosal depth.

In an embodiment, the flora-sampling module 130 includes one or more articulated structure elements. In an embodiment, the sample-collection assembly 304 includes one or more telescopic structural elements. In an embodiment, the sample-collection assembly 304 includes one or more retractable structural elements, telescoping elements, or articulating elements. In an embodiment, the sample-collection assembly 304 includes one or more extendible structural elements.

In an embodiment, the sample-collection assembly 304 includes one or more articulated collection probes. In an embodiment, the sample-collection assembly 304 includes one or more telescopic collection probes. In an embodiment, the sample-collection assembly 304 includes one or more retractable collection probes. In an embodiment, the sample-collection assembly 304 includes one or more extendible collection probes. In an embodiment, the sample-collection assembly 304 includes one or more collection probes. In an embodiment, the sample-collection assembly 304 includes one or more articulated structure configured to scrape away matter from a biological surface. In an embodiment, the sample-collection assembly 304 is configured to provide fluidic communication between an interior environment of the electronic suppository 101 and an exterior environment, and configured to collect an intestinal microbial flora sample

In an embodiment, the flora-mapping module 152 includes circuitry configured to generate flora collection information associated with collection of at least one intestinal microbial flora sample. In an embodiment, the flora-mapping module 152 includes circuitry configured to generate flora collection information responsive to a microbial flora collection event. In an embodiment, the flora-mapping module 152 includes circuitry configured to wirelessly transmit flora collection information responsive to a microbial flora collection event. In an embodiment, the flora-mapping module 152 includes circuitry configured to wirelessly transmit flora collection information responsive to a target schedule. In an embodiment, the flora-mapping module 152 includes circuitry configured to wirelessly transmit flora collection information responsive to an input indicative of target collection site.

In an embodiment, the flora-mapping module 152 includes circuitry configured to wirelessly transmit flora collection information responsive to an input indicative of request for transmission. In an embodiment, the flora-mapping module 152 includes circuitry configured to wirelessly transmit flora collection information responsive to a query from a remote enterprise. In an embodiment, the flora-mapping module 152 includes circuitry configured to wirelessly transmit flora collection information responsive to interrogation energy satisfying a threshold criterion.

In an embodiment, the flora-mapping module 152 includes circuitry configured to toggle between a transmit state and a receive state upon electromagnetic energy interrogation satisfying a threshold criterion. In an embodiment, the flora-mapping module 152 includes an interrogation interface operable to transmit flora collection information responsive to interrogation energy satisfying a threshold criterion. In an embodiment, the flora-mapping module 152 includes circuitry configured to actuate a discovery protocol that allows the flora-mapping module and a remote enterprise to identify each other and to negotiate one or more pre-shared keys. In an embodiment, the flora-mapping module 152 includes circuitry configured to transmit or receive user-specific flora collection information based on at least one of an authorization protocol, an authentication protocol, or an activation protocol.

In an embodiment, the egress module 102 includes circuitry configured to actuate an egress process responsive to a target schedule. In an embodiment, the egress module 102 includes circuitry configured to actuate an egress process responsive to at least one input indicative of a peristaltic reflex process. In an embodiment, the egress module 102 includes circuitry configured to actuate an egress process responsive to at least one input indicative of an onset of a defecation event.

In an embodiment, the egress module 102 includes circuitry configured to actuate an egress process responsive to a comparison, between the flora collection information and user-specific microbial flora-sampling information, indicative of a collection event. In an embodiment, the egress module 102 includes circuitry configured to actuate an egress process according to a programmable schedule. In an embodiment, the egress module 102 includes circuitry configured to communicate an onset of an egress event. In an embodiment, the egress module 102 includes circuitry having an interrogation interface configured to communicate an onset of an egress even responsive to interrogation of the interrogation interface.

In an embodiment, the ingestible flora-sampling device 301 includes an extendable mechanism having structural elements configured contact two or more portion of an intestinal wall. For example, in an embodiment, the ingestible flora-sampling device 301 includes an extendable mechanism having structural elements having one or more mechanical legs configured to contact two or more portion of an intestinal wall. In an embodiment, the structural elements include one or more inflatable elements. In an embodiment, the structural elements include one or more bimorphs (e.g., bimorph actuators, bimorph cantilevers, piezoelectric bimorphs, etc.).

In an embodiment, the ingestible flora-sampling device 301 includes an extendable mechanism having structural elements configured to physically contact two or more portion of an intestinal wall and to frictionally couple the ingestible flora-sampling device to an intestinal wall. In an embodiment, the ingestible flora-sampling device 301 includes an extendable mechanism having an inchworm motor device that enables the, the ingestible flora-sampling device 301 to move along the wall of an intestinal tract.

In an embodiment, the ingestible flora-sampling device 301 includes an extendable mechanism having structural elements configured to frictionally couple the ingestible flora-sampling device to an intestinal wall during collection of at least one intestinal microbial flora sample.

In an embodiment, the ingestible flora-sampling device 301 includes a flora-sampling module having circuitry configured to actuate collection of at least one intestinal microbial flora sample and a flora-mapping module having circuitry configured to generate flora collection information associated with collection of at least one intestinal microbial flora sample.

In an embodiment, the ingestible flora-sampling device 301 includes an extendable mechanism having structural elements configured contact two or more portion of an intestinal wall. In an embodiment, the structural elements include one or more mechanical legs. In an embodiment, the structural elements include one or more inflatable elements. In an embodiment, the structural elements include one or more bimorphs (e.g., bimorph actuators, bimorph cantilevers, piezoelectric bimorphs, etc.). In an embodiment, the extendable mechanism includes an inchworm motor device.

In an embodiment, the ingestible flora-sampling device 301 includes an extendable mechanism having structural elements configured to physically contact two or more portion of an intestinal wall and to frictionally couple the ingestible flora-sampling device to an intestinal wall. In an embodiment, the ingestible flora-sampling device 301 includes an extendable mechanism having structural elements configured to frictionally couple the ingestible flora-sampling device to an intestinal wall during collection of at least one intestinal microbial flora sample.

In an embodiment the ingestible flora-sampling device 301 include one or more structures configure to enable the ingestible flora-sampling device 301 to collect samples at various depths of the mucosa, at various locations away from the surface the ingestible flora-sampling device 301, or the like. For example, in an embodiment, a sample-collection assembly 304 includes one or more articulated collection structure having fluid-flow passages within for transporting a sample (e.g., biological sample, a microbial flora sample, fluid samples from intestinal mucosa, or the like) from an exterior environment to an interior environment of the ingestible flora-sampling device 301. In an embodiment, the sample-collection assembly 304 includes one or more telescopic collection structures. In an embodiment, the sample-collection assembly 304 includes one or more retractable collection structures. In an embodiment, the sample-collection assembly 304 includes one or more extendible collection structures.

In an embodiment, the sample-collection assembly 304 includes one or more articulated structure configured to scrape away matter form a biological surface.

Referring to FIGS. 4A and 4B, in an embodiment, an article of manufacture 402, an apparatus (e.g., a medical apparatus, etc.), or the like, includes a signal-bearing medium bearing one or more instruction for causing a computing device to control an egress process associated with egress of an electronic suppository from an intestinal tract. In an embodiment, an article of manufacture 402 includes a signal-bearing medium bearing one or more instruction for causing a computing device to actuate electrical stimulation of a region of intestinal mucosal surface proximate the electronic suppository. In an embodiment, an article of manufacture 402 includes one or more instruction for causing a computing device to actuate one or more transducers configured to acoustically stimulate a region of intestinal mucosal surface proximate the electronic suppository.

In an embodiment, an article of manufacture 402 includes one or more instruction for causing a computing device to actuate one or more surface contact elements associated with an electronic suppository to physically contact one or more regions of intestinal mucosal surface proximate the electronic suppository and to mechanically stimulate one or more regions of intestinal mucosal surface. In an embodiment, an article of manufacture 402 includes one or more instruction for causing a computing device to actuate a plurality of surface contact elements 124 configured to physically distend one or more regions of intestinal mucosal surface. In an embodiment, an article of manufacture 402 includes one or more instruction for causing a computing device to actuate an egress process associated with egress of an electronic suppository responsive to at least one input indicative of an onset of a defecation event.

In an embodiment, an article of manufacture 402 includes one or more instruction for causing a computing device to actuate an egress process associated with egress of an electronic suppository responsive to at least one input indicative of a peristaltic contraction in an intestinal tract. In an embodiment, an article of manufacture 402 includes one or more instruction for causing a computing device to actuate an egress process associated with egress of an electronic suppository responsive to at least one input indicative of a peristaltic reflex process. In an embodiment, an article of manufacture 402 includes one or more instruction for causing a computing device to actuate an egress process associated with egress of an electronic suppository responsive to a time-based protocol.

In an embodiment, an article of manufacture 402 includes one or more instruction for causing a computing device to actuate an egress process associated with egress of an electronic suppository responsive to target schedule information. In an embodiment, an article of manufacture 402 includes one or more instruction for causing a computing device to actuate an egress process associated with egress of an electronic suppository responsive to at least one measurand from one or more sensors 154 indicative of a contraction of one or more expiratory chest muscles. In an embodiment, an article of manufacture 402 includes one or more instruction for causing a computing device to actuate an egress process associated with egress of an electronic suppository responsive to at least one measurand from one or more sensors 154 indicative of a diaphragm contraction. In an embodiment, an article of manufacture 402 includes one or more instruction for causing a computing device to actuate an egress process associated with egress of an electronic suppository responsive to at least one measurand from one or more sensors 154 indicative of a contraction of one or more abdominal wall muscles.

Referring to FIG. 4B, in an embodiment, an article of manufacture 402 includes one or more instruction for causing a computing device to actuate an egress process associated with egress of an electronic suppository responsive to at least one measurand from one or more sensors 154 indicative of a contraction of a pelvic diaphragm. In an embodiment, an article of manufacture 402 includes one or more instruction for causing a computing device to actuate an egress process associated with egress of an electronic suppository responsive to at least one measurand from one or more sensors 154 indicative of a change in pressure on an intestinal tract.

In an embodiment, an article of manufacture 402 includes one or more instruction for causing a computing device to actuate an egress process associated with egress of an electronic suppository responsive to at least one measurand from one or more sensors 154 indicative of a presence of one or more peristaltic waves. In an embodiment, an article of manufacture 402 includes one or more instruction for causing a computing device to actuate an egress process associated with egress of an electronic suppository responsive to at least one measurand from one or more sensors 154 indicative of peristaltic contractions in an intestinal tract. In an embodiment, an article of manufacture 402 includes one or more instruction for causing a computing device to actuate delivery of at least one lubricating material from one or more reservoirs of an electronic suppository, to an outer surface of the electronic suppository.

In an embodiment, an article of manufacture 402 includes one or more instruction for causing a computing device to actuate delivery of at least one laxative composition from one or more reservoirs of an electronic suppository, to an outer surface of the electronic suppository. In an embodiment, an article of manufacture 452 includes one or more instruction for causing a computing device to actuate a change in a physical dimension of an electronic suppository to facilitate egress of the electronic suppository from an intestinal tract.

Referring to FIG. 5, in an embodiment, an article of manufacture 502, an apparatus (e.g., a medical apparatus, etc.), or the like, includes a signal-bearing medium bearing a non-transitory signal-bearing medium bearing one or more instruction for causing a computing device to registering at least a first intestinal anatomical target along an intestinal tract to user-specific intestinal registration information. In an embodiment, an article of manufacture 502 includes one or more instruction for causing a computing device to deliver, via an electronic suppository, a microbial flora composition to the first intestinal anatomical target. In an embodiment, an article of manufacture 502 includes one or more instruction for causing a computing device to register at least a second intestinal anatomical target.

In an embodiment, an article of manufacture 502 includes one or more instruction for causing a computing device to deliver, via the electronic suppository, a microbial flora composition to the second anatomical target. In an embodiment, an article of manufacture 502 includes one or more instruction for causing a computing device to generate microbial flora delivery registration information associated with delivery of the microbial flora composition to the first intestinal anatomical target.

In an embodiment, an article of manufacture 502 includes one or more instruction for causing a computing device to generate microbial flora delivery registration information associated with delivery of the microbial flora composition to the first intestinal anatomical target. In an embodiment, the microbial flora delivery registration information includes one or more of an intestinal anatomical target identification, intestinal anatomical target location, an intestinal anatomical target shape, an intestinal anatomical target dimension, an intestinal anatomical target distribution, and a point cloud associated with an intestinal anatomical target. In an embodiment, an article of manufacture 502 includes one or more instruction for causing a computing device to register a plurality of intestinal anatomical targets. In an embodiment, an article of manufacture 502 includes one or more instruction for causing a computing device to generate a first-in-time microbial flora composition delivery target and a second-in-time microbial flora composition delivery target responsive to registering the plurality of intestinal anatomical targets.

FIG. 6 shows a method 600 of delivering microbial flora within an intestinal tract. At 610, the method 600 includes registering at least a first intestinal anatomical target along an intestinal tract to user-specific intestinal registration information. At 612, registering the first intestinal anatomical target includes determining a plurality of references points along the intestinal tract and registering the first intestinal anatomical target to the plurality of references points. At 614, registering the first intestinal anatomical target includes determining detecting at least one anatomical feature along the intestinal tract and registering the first intestinal anatomical target to the at least one anatomical feature along the intestinal tract. At 620, the method 600 includes delivering, via an electronic suppository, a microbial flora composition to the first intestinal anatomical target. At 622, delivering the microbial flora composition to the first plurality of anatomical targets includes delivering the microbial flora composition responsive to one or more patient-specific instructions. In an embodiment, delivering the microbial flora composition to the first plurality of anatomical targets includes delivering the microbial flora composition responsive to one or more measurands indicative of a target location.

At 630, the method 600 includes registering at least a second intestinal anatomical target to user-specific intestinal registration information. At 640, the method 600 includes delivering, via the electronic suppository, a microbial flora composition to the second anatomical target. At 660, the method 600 includes generating microbial flora delivery registration information associated with delivery of the microbial flora composition to the first intestinal anatomical target. At 660, the method 600 includes generating microbial flora delivery registration information associated with delivery of the microbial flora composition to the first intestinal anatomical target. In an embodiment, generating microbial flora delivery registration information includes generating one or more of an intestinal anatomical target identification, intestinal anatomical target location, an intestinal anatomical target shape, an intestinal anatomical target dimension, an intestinal anatomical target distribution, and a point cloud associated with an intestinal anatomical target. At 670, the method 600 includes registering a plurality of intestinal anatomical targets. At 680, the method 600 includes generating a first-in-time microbial flora composition delivery target and a second-in-time microbial flora composition delivery target responsive to registering the plurality of intestinal anatomical targets.

It is noted that FIG. 6 denotes “start” and “end” positions. However, nothing herein should be construed to indicate that these are limiting and it is contemplated that other or additional steps or functions can occur before or after those described in FIG. 6.

The claims, description, and drawings of this application may describe one or more of the instant technologies in operational/functional language, for example as a set of operations to be performed by a computer. Such operational/functional description in most instances can be specifically-configured hardware (e.g., because a general purpose computer in effect becomes a special purpose computer once it is programmed to perform particular functions pursuant to instructions from program software).

Importantly, although the operational/functional descriptions described herein are understandable by the human mind, they are not abstract ideas of the operations/functions divorced from computational implementation of those operations/functions. Rather, the operations/functions represent a specification for the massively complex computational machines or other means. As discussed in detail below, the operational/functional language must be read in its proper technological context, i.e., as concrete specifications for physical implementations.

The logical operations/functions described herein are a distillation of machine specifications or other physical mechanisms specified by the operations/functions such that the otherwise inscrutable machine specifications may be comprehensible to the human mind. The distillation also allows one of skill in the art to adapt the operational/functional description of the technology across many different specific vendors' hardware configurations or platforms, without being limited to specific vendors' hardware configurations or platforms.

Some of the present technical description (e.g., detailed description, drawings, claims, etc.) may be set forth in terms of logical operations/functions. As described in more detail in the following paragraphs, these logical operations/functions are not representations of abstract ideas, but rather representative of static or sequenced specifications of various hardware elements. Differently stated, unless context dictates otherwise, the logical operations/functions are representative of static or sequenced specifications of various hardware elements. This is true because tools available to implement technical disclosures set forth in operational/functional formats—tools in the form of a high-level programming language (e.g., C, java, visual basic), etc.), or tools in the form of Very high speed Hardware Description Language (“VHDL,” which is a language that uses text to describe logic circuits-)—are generators of static or sequenced specifications of various hardware configurations. This fact is sometimes obscured by the broad term “software,” but, as shown by the following explanation, what is termed “software” is a shorthand for a massively complex interchaining/specification of ordered-matter elements. The term “ordered-matter elements” may refer to physical components of computation, such as assemblies of electronic logic gates, molecular computing logic constituents, quantum computing mechanisms, etc.

For example, a high-level programming language is a programming language with strong abstraction, e.g., multiple levels of abstraction, from the details of the sequential organizations, states, inputs, outputs, etc., of the machines that a high-level programming language actually specifies. See, e.g., Wikipedia, High-level programming language, http://en.wikipedia.org/wiki/High-level_programming_language (as of Jun. 5, 2012, 21:00 GMT). In order to facilitate human comprehension, in many instances, high-level programming languages resemble or even share symbols with natural languages. See, e.g., Wikipedia, Natural language, http://en.wikipedia.org/wiki/Natural_language (as of Jun. 5, 2012, 21:00 GMT).

It has been argued that because high-level programming languages use strong abstraction (e.g., that they may resemble or share symbols with natural languages), they are therefore a “purely mental construct.” (e.g., that “software”—a computer program or computer-programming—is somehow an ineffable mental construct, because at a high level of abstraction, it can be conceived and understood in the human mind). This argument has been used to characterize technical description in the form of functions/operations as somehow “abstract ideas.” In fact, in technological arts (e.g., the information and communication technologies) this is not true.

The fact that high-level programming languages use strong abstraction to facilitate human understanding should not be taken as an indication that what is expressed is an abstract idea. In an embodiment, if a high-level programming language is the tool used to implement a technical disclosure in the form of functions/operations, it can be understood that, far from being abstract, imprecise, “fuzzy,” or “mental” in any significant semantic sense, such a tool is instead a near incomprehensibly precise sequential specification of specific computational—machines—the parts of which are built up by activating/selecting such parts from typically more general computational machines over time (e.g., clocked time). This fact is sometimes obscured by the superficial similarities between high-level programming languages and natural languages. These superficial similarities also may cause a glossing over of the fact that high-level programming language implementations ultimately perform valuable work by creating/controlling many different computational machines.

The many different computational machines that a high-level programming language specifies are almost unimaginably complex. At base, the hardware used in the computational machines typically consists of some type of ordered matter (e.g., traditional electronic devices (e.g., transistors), deoxyribonucleic acid (DNA), quantum devices, mechanical switches, optics, fluidics, pneumatics, optical devices (e.g., optical interference devices), molecules, etc.) that are arranged to form logic gates. Logic gates are typically physical devices that may be electrically, mechanically, chemically, or otherwise driven to change physical state in order to create a physical reality of Boolean logic.

Logic gates may be arranged to form logic circuits, which are typically physical devices that may be electrically, mechanically, chemically, or otherwise driven to create a physical reality of certain logical functions. Types of logic circuits include such devices as multiplexers, registers, arithmetic logic units (ALUs), computer memory devices, etc., each type of which may be combined to form yet other types of physical devices, such as a central processing unit (CPU)—the best known of which is the microprocessor. A modern microprocessor will often contain more than one hundred million logic gates in its many logic circuits (and often more than a billion transistors). See, e.g., Wikipedia, Logic gates, http://en.wikipedia.org/wiki/Logic_gates (as of Jun. 5, 2012, 21:03 GMT).

The logic circuits forming the microprocessor are arranged to provide a microarchitecture that will carry out the instructions defined by that microprocessor's defined Instruction Set Architecture. The Instruction Set Architecture is the part of the microprocessor architecture related to programming, including the native data types, instructions, registers, addressing modes, memory architecture, interrupt and exception handling, and external Input/Output. See, e.g., Wikipedia, Computer architecture, http://en.wikipedia.org/wiki/Computer_architecture (as of Jun. 5, 2012, 21:03 GMT).

The Instruction Set Architecture includes a specification of the machine language that can be used by programmers to use/control the microprocessor. Since the machine language instructions are such that they may be executed directly by the microprocessor, typically they consist of strings of binary digits, or bits. For example, a typical machine language instruction might be many bits long (e.g., 32, 64, or 128 bit strings are currently common). A typical machine language instruction might take the form “11110000101011110000111100111111” (a 32 bit instruction).

It is significant here that, although the machine language instructions are written as sequences of binary digits, in actuality those binary digits specify physical reality. For example, if certain semiconductors are used to make the operations of Boolean logic a physical reality, the apparently mathematical bits “1” and “0” in a machine language instruction actually constitute a shorthand that specifies the application of specific voltages to specific wires. For example, in some semiconductor technologies, the binary number “1” (e.g., logical “1”) in a machine language instruction specifies around +5 volts applied to a specific “wire” (e.g., metallic traces on a printed circuit board) and the binary number “0” (e.g., logical “0”) in a machine language instruction specifies around −5 volts applied to a specific “wire.” In addition to specifying voltages of the machines' configuration, such machine language instructions also select out and activate specific groupings of logic gates from the millions of logic gates of the more general machine. Thus, far from abstract mathematical expressions, machine language instruction programs, even though written as a string of zeros and ones, specify many, many constructed physical machines or physical machine states.

Machine language is typically incomprehensible by most humans (e.g., the above example was just ONE instruction, and some personal computers execute more than two billion instructions every second). See, e.g., Wikipedia, Instructions per second, http://en.wikipedia.org/wiki/Instructions_per_second (as of Jun. 5, 2012, 21:04 GMT).

Thus, programs written in machine language—which may be tens of millions of machine language instructions long—are incomprehensible. In view of this, early assembly languages were developed that used mnemonic codes to refer to machine language instructions, rather than using the machine language instructions' numeric values directly (e.g., for performing a multiplication operation, programmers coded the abbreviation “mult,” which represents the binary number “011000” in MIPS machine code). While assembly languages were initially a great aid to humans controlling the microprocessors to perform work, in time the complexity of the work that needed to be done by the humans outstripped the ability of humans to control the microprocessors using merely assembly languages.

At this point, it was noted that the same tasks needed to be done over and over, and the machine language necessary to do those repetitive tasks was the same. In view of this, compilers were created. A compiler is a device that takes a statement that is more comprehensible to a human than either machine or assembly language, such as “add 2+2 and output the result,” and translates that human understandable statement into a complicated, tedious, and immense machine language code (e.g., millions of 32, 64, or 128 bit length strings). Compilers thus translate high-level programming language into machine language.

This compiled machine language, as described above, is then used as the technical specification which sequentially constructs and causes the interoperation of many different computational machines such that humanly useful, tangible, and concrete work is done. For example, as indicated above, such machine language—the compiled version of the higher-level language—functions as a technical specification which selects out hardware logic gates, specifies voltage levels, voltage transition timings, etc., such that the humanly useful work is accomplished by the hardware.

Thus, a functional/operational technical description, when viewed by one of skill in the art, is far from an abstract idea. Rather, such a functional/operational technical description, when understood through the tools available in the art such as those just described, is instead understood to be a humanly understandable representation of a hardware specification, the complexity and specificity of which far exceeds the comprehension of most any one human. Accordingly, any such operational/functional technical descriptions may be understood as operations made into physical reality by (a) one or more interchained physical machines, (b) interchained logic gates configured to create one or more physical machine(s) representative of sequential/combinatorial logic(s), (c) interchained ordered matter making up logic gates (e.g., interchained electronic devices (e.g., transistors), DNA, quantum devices, mechanical switches, optics, fluidics, pneumatics, molecules, etc.) that create physical reality representative of logic(s), or (d) virtually any combination of the foregoing. Indeed, any physical object which has a stable, measurable, and changeable state may be used to construct a machine based on the above technical description. Charles Babbage, for example, constructed the first computer out of wood and powered by cranking a handle.

Thus, far from being understood as an abstract idea, it can be recognizes that a functional/operational technical description as a humanly-understandable representation of one or more almost unimaginably complex and time sequenced hardware instantiations. The fact that functional/operational technical descriptions might lend themselves readily to high-level computing languages (or high-level block diagrams for that matter) that share some words, structures, phrases, etc. with natural language simply cannot be taken as an indication that such functional/operational technical descriptions are abstract ideas, or mere expressions of abstract ideas. In fact, as outlined herein, in the technological arts this is simply not true. When viewed through the tools available to those of skill in the art, such functional/operational technical descriptions are seen as specifying hardware configurations of almost unimaginable complexity.

As outlined above, the reason for the use of functional/operational technical descriptions is at least twofold. First, the use of functional/operational technical descriptions allows near-infinitely complex machines and machine operations arising from interchained hardware elements to be described in a manner that the human mind can process (e.g., by mimicking natural language and logical narrative flow). Second, the use of functional/operational technical descriptions assists the person of skill in the art in understanding the described subject matter by providing a description that is more or less independent of any specific vendor's piece(s) of hardware.

The use of functional/operational technical descriptions assists the person of skill in the art in understanding the described subject matter since, as is evident from the above discussion, one could easily, although not quickly, transcribe the technical descriptions set forth in this document as trillions of ones and zeroes, billions of single lines of assembly-level machine code, millions of logic gates, thousands of gate arrays, or any number of intermediate levels of abstractions. However, if any such low-level technical descriptions were to replace the present technical description, a person of skill in the art could encounter undue difficulty in implementing the disclosure, because such a low-level technical description would likely add complexity without a corresponding benefit (e.g., by describing the subject matter utilizing the conventions of one or more vendor-specific pieces of hardware). Thus, the use of functional/operational technical descriptions assists those of skill in the art by separating the technical descriptions from the conventions of any vendor-specific piece of hardware.

In view of the foregoing, the logical operations/functions set forth in the present technical description are representative of static or sequenced specifications of various ordered-matter elements, in order that such specifications may be comprehensible to the human mind and adaptable to create many various hardware configurations. The logical operations/functions disclosed herein should be treated as such, and should not be disparagingly characterized as abstract ideas merely because the specifications they represent are presented in a manner that one of skill in the art can readily understand and apply in a manner independent of a specific vendor's hardware implementation.

At least a portion of the devices or processes described herein can be integrated into an information processing system. An information processing system generally includes one or more of a system unit housing, a video display device, memory, such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), or control systems including feedback loops and control motors (e.g., feedback for detecting position or velocity, control motors for moving or adjusting components or quantities). An information processing system can be implemented utilizing suitable commercially available components, such as those typically found in data computing/communication or network computing/communication systems.

The state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Various vehicles by which processes or systems or other technologies described herein can be effected (e.g., hardware, software, firmware, etc., in one or more machines or articles of manufacture), and that the preferred vehicle will vary with the context in which the processes, systems, other technologies, etc., are deployed. For example, if an implementer determines that speed and accuracy are paramount, the implementer may opt for a mainly hardware or firmware vehicle; alternatively, if flexibility is paramount, the implementer may opt for a mainly software implementation that is implemented in one or more machines or articles of manufacture; or, yet again alternatively, the implementer may opt for some combination of hardware, software, firmware, etc. in one or more machines or articles of manufacture. Hence, there are several possible vehicles by which the processes, devices, other technologies, etc., described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. In an embodiment, optical aspects of implementations will typically employ optically-oriented hardware, software, firmware, etc., in one or more machines or articles of manufacture.

The herein described subject matter sometimes illustrates different components contained within, or connected with, different other components. It is to be understood that such depicted architectures are merely examples, and that in fact, many other architectures can be implemented that achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably coupleable,” to each other to achieve the desired functionality. Specific examples of operably coupleable include, but are not limited to, physically mateable, physically interacting components, wirelessly interactable, wirelessly interacting components, logically interacting, logically interactable components, etc.

In an embodiment, one or more components may be referred to herein as “configured to,” “configurable to,” “operable/operative to,” “adapted/adaptable,” “able to,” “conformable/conformed to,” etc. Such terms (e.g., “configured to”) can generally encompass active-state components, or inactive-state components, or standby-state components, unless context requires otherwise.

The foregoing detailed description has set forth various embodiments of the devices or processes via the use of block diagrams, flowcharts, or examples. Insofar as such block diagrams, flowcharts, or examples contain one or more functions or operations, it will be understood by the reader that each function or operation within such block diagrams, flowcharts, or examples can be implemented, individually or collectively, by a wide range of hardware, software, firmware in one or more machines or articles of manufacture, or virtually any combination thereof. Further, the use of “Start,” “End,” or “Stop” blocks in the block diagrams is not intended to indicate a limitation on the beginning or end of any functions in the diagram. Such flowcharts or diagrams may be incorporated into other flowcharts or diagrams where additional functions are performed before or after the functions shown in the diagrams of this application. In an embodiment, several portions of the subject matter described herein is implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal-bearing medium used to actually carry out the distribution. Non-limiting examples of a signal-bearing medium include the following: a recordable type medium such as a floppy disk, a hard disk drive, a Compact Disc (CD), a Digital Video Disk (DVD), a digital tape, a computer memory, etc.; and a transmission type medium such as a digital or an analog communication medium (e.g., a fiber optic cable, a waveguide, a wired communications link, a wireless communication link (e.g., transmitter, receiver, transmission logic, reception logic, etc.), etc.).

While particular aspects of the present subject matter described herein have been shown and described, it will be apparent to the reader that, based upon the teachings herein, changes and modifications can be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Further, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense of the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense of the convention (e.g., “a system having at least one of A, B, or C” would include but not be limited to systems that have A alone, B alone, C alone, A and B together, A and C together, B and C together, and/or A, B, and C together, etc.). Typically a disjunctive word or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, the operations recited therein generally may be performed in any order. Also, although various operational flows are presented in a sequence(s), it should be understood that the various operations may be performed in orders other than those that are illustrated, or may be performed concurrently. Examples of such alternate orderings includes overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

1. An electronic suppository system, comprising:
an electronic suppository egress module including circuitry configured to control an egress process associated with egress of an electronic suppository from an intestinal tract.

2. The electronic suppository system of claim 1, further comprising:
an intestinal peristalsis induction module operably coupled to the electronic suppository egress module, the intestinal peristalsis induction module having circuitry configured to electrically stimulate a region of intestinal mucosal surface.

3. The electronic suppository system of claim 1, further comprising:
an intestinal peristalsis induction module operably coupled to the electronic suppository egress module, the intestinal peristalsis induction module having one or more transducers configured to acoustically stimulate a region of intestinal mucosal surface.

4. The electronic suppository system of claim 1, further comprising:
an intestinal peristalsis induction module operably coupled to the electronic suppository egress module, the intestinal peristalsis induction module having one or more surface contact elements configured to physically contact one or more regions of intestinal mucosal surface and to mechanically stimulate a region of intestinal mucosal surface.

5. The electronic suppository system of claim 1, further comprising:
a communication module including at least one of a receiver, a transmitter, and a transceiver operable to report intent to egress.

6. The electronic suppository system of claim 1, further comprising:
a communication module including at least one of a receiver, a transmitter, and a transceiver operable to report activation of egress.

7.-9. (canceled)

10. The electronic suppository system of claim 1, further comprising:
an intestinal peristalsis induction module operably coupled to the electronic suppository egress module, the intestinal peristalsis induction module having a plurality of surface contact elements configured to physically distend one or more regions of intestinal mucosal surface.

11.-14. (canceled)

15. The electronic suppository system of claim 1, wherein the electronic suppository egress module includes circuitry configured to actuate an egress process associated with egress of an electronic suppository responsive to at least one input indicative of an onset of a defecation event.

16.-20. (canceled)

21. The electronic suppository system of claim 1, wherein the electronic suppository egress module includes circuitry configured to actuate an egress process associated with egress of an electronic suppository responsive to at least one measurand indicative of a contraction of one or more abdominal wall muscles.

22.-23. (canceled)

24. The electronic suppository system of claim 1, wherein the electronic suppository egress module includes circuitry configured to actuate an egress process associated with egress of an electronic suppository responsive to at least one measurand indicative of one or more peristaltic waves.

25. The electronic suppository system of claim 1, wherein the electronic suppository egress module includes circuitry configured to actuate an egress process associated with egress of an electronic suppository responsive to at least one measurand indicative of peristaltic contractions in an intestinal tract.

26. The electronic suppository system of claim 1, further comprising:
a lubricant supply component operably coupled to the electronic suppository egress module, the lubricant supply component including one or more reservoirs and circuitry configured to activate delivery of at least one lubricating material from the one or more reservoirs, to an outer surface on an electronic suppository.

27. The electronic suppository system of claim 26, wherein the electronic suppository egress module includes circuitry configured to actuate delivery of at least one lubricating material responsive to activation of an egress protocol associated with egress of an electronic suppository from an intestinal tract.

28. The electronic suppository system of claim 1, further comprising:
a laxative supply module coupled to the electronic suppository egress module, the laxative supply module configured to deliver at least one laxative composition to an outer surface on an electronic suppository

29. (canceled)

30. The electronic suppository system of claim 1, further comprising:
an electronic suppository egress module including circuitry configured to actuate a change in a physical dimension of an electronic suppository to facilitate egress of the electronic suppository from an intestinal tract.

31. The electronic suppository system of claim 1, further comprising:
a defecation detection module operably coupled to the electronic suppository egress module, the defecation detection module having circuitry configured to generate real-time defecation event information responsive to at least on measurand indicative of a defecation event.

32. The electronic suppository system of claim 1, further comprising:
a defecation detection module operably coupled to the electronic suppository egress module, the defecation detection module having circuitry configured to generate real-time defecation event information responsive to receipt of one or more user commands.

33. (canceled)

34. The electronic suppository system of claim 1, further comprising:
a defecation detection module operably coupled to the electronic suppository egress module, the defecation detection module having circuitry configured to toggle between and activated state and an inactivated state responsive to wireless receipt of one or more egress commands.

35. The electronic suppository system of claim 1, further comprising:
a flora-sampling module including circuitry configured to actuate the collection of one or more intestinal microbial flora samples.

36. (canceled)

37. The electronic suppository system of claim 35, wherein the flora-sampling module includes circuitry configured to actuate the collection of the one or more intestinal microbial flora samples responsive to time-since-meal information.

38. The electronic suppository system of claim 35, wherein the flora-sampling module includes circuitry operably coupled to a collection-site scraper assembly, and configured to actuate the scraping of a collection site of prior to collection of the one or more intestinal microbial flora samples.

39. An article of manufacture, comprising:
a non-transitory signal-bearing medium bearing
one or more instruction for causing a computing device to control an egress process associated with egress of an electronic suppository from an intestinal tract.

40. The article of manufacture 39, further comprising:
one or more instruction for causing a computing device to actuate electrical stimulation of a region of intestinal mucosal surface proximate the electronic suppository.

41. The article of manufacture 39, further comprising:
one or more instruction for causing a computing device to actuate one or more transducers configured to acoustically stimulate a region of intestinal mucosal surface proximate the electronic suppository.

42. The article of manufacture 39, further comprising:
one or more instruction for causing a computing device to actuate one or more surface contact elements associated with an electronic suppository to physically contact one or more regions of intestinal mucosal surface proximate the electronic suppository and to mechanically stimulate one or more regions of intestinal mucosal surface.

43. The article of manufacture 39, further comprising:
one or more instruction for causing a computing device to actuate a plurality of surface contact elements configured to physically distend one or more regions of intestinal mucosal surface.

44.-45. (canceled)

46. The article of manufacture 39, further comprising:
one or more instruction for causing a computing device to actuate an egress process associated with egress of an electronic suppository responsive to at least one input indicative of a peristaltic reflex process.

47. The article of manufacture 39, further comprising:
one or more instruction for causing a computing device to actuate an egress process associated with egress of an electronic suppository responsive to a time-based protocol.

48. The article of manufacture 39, further comprising:
one or more instruction for causing a computing device to actuate an egress process associated with egress of an electronic suppository responsive to a target schedule information.

49.-50. (canceled)

51. The article of manufacture 39, further comprising:
one or more instruction for causing a computing device to actuate an egress process associated with egress of an electronic suppository responsive to at least one measurand indicative of a contraction of one or more abdominal wall muscles.

52. The article of manufacture 39, further comprising:
one or more instruction for causing a computing device to actuate an egress process associated with egress of an electronic suppository responsive to at least one measurand indicative of a contraction of a pelvic diaphragm.

53. The article of manufacture 39, further comprising:
one or more instruction for causing a computing device to actuate an egress process associated with egress of an electronic suppository responsive to at least one measurand indicative of a change in pressure on an intestinal tract.

54. The article of manufacture 39, further comprising:
one or more instruction for causing a computing device to actuate an egress process associated with egress of an electronic suppository responsive to at least one measurand indicative of a presence of one or more peristaltic waves.

55. The article of manufacture 39, further comprising:
one or more instruction for causing a computing device to actuate an egress process associated with egress of an electronic suppository responsive to at least one measurand indicative of peristaltic contractions in an intestinal tract.

56. The article of manufacture 39, further comprising:
one or more instruction for causing a computing device to actuate delivery of at least one lubricating material from one or more reservoirs of an electronic suppository, to an outer surface of the electronic suppository.

57. The article of manufacture 39, further comprising:
one or more instruction for causing a computing device to actuate delivery of at least one laxative composition from one or more reservoirs of an electronic suppository, to an outer surface of the electronic suppository.

58. The article of manufacture 39, further comprising:
one or more instruction for causing a computing device to actuate a change in a physical dimension of an electronic suppository to facilitate egress of the electronic suppository from an intestinal tract.

59.-201. (canceled)
